(19) United States
(12) Patent Application Publication
(10) Pub. No.: US 2026/0123985 A1
(43) Pub. Date: May 7, 2026

(54) MULTIPLE ELECTRODE HEART VALVE SLITTING DEVICE

(71) Applicant: KONINKLIJKE PHILIPS N.V., EINDHOVEN (NL)

(72) Inventors: Mikhail Borisovich TIKH, ST. LOUIS PARK, MN (US); James Allen JENSEN, MAPLE GROVE, MN (US)

(21) Appl. No.: 19/346,913

(22) Filed: Oct. 1, 2025

Related U.S. Application Data
(60) Provisional application No. 63/715,677, filed on Nov. 4, 2024.

(51) Int. Cl. A61B 18/14; A61B 18/00

(52) U.S. Cl. CPC A61B 18/1492; A61B 2018/00184; A61B 2018/00369; A61B 2018/00601; A61B 2018/144

(57) ABSTRACT

An apparatus includes a flexible elongate member positionable within a patient body, and including a first tine having a first crossing electrode and a first cutting electrode, a second tine having a second crossing electrode and a second cutting electrode; and a polymer jacket positioned around the first tine and the second tine. The polymer jacket is configured to prevent longitudinal and rotational motion of the first tine and second tines relative to one another. The first tine and second tines are configured to transition between a straight configuration and a hook-shaped configuration. The first crossing electrode and second crossing electrode are configured to cut anatomy of the patient body while the first tine and second tine are in the straight configuration. The first cutting electrode and second cutting electrode are configured to cut the anatomy while the first tine and second tine are tine are in the hook-shaped configuration.

### TECHNICAL FIELD

The subject matter described herein relates to cardiac valve replacement and, in particular, to the reducing or eliminating blood flow obstruction to the human heart before/during cardiac valve replacement procedures (e.g., to prepare the site for valve replacement). For example, a catheter-deployed, multi-tined intravascular leaflet slitting device may be utilized to produce multiple simultaneous slits in a heart valve (e.g., aortic valve) leaflet, to prevent obstruction of coronary arteries.

### BACKGROUND

One common type of valve disease is aortic valve stenosis, in which the valve between the aorta and the heart’s left ventricle is narrowed and doesn't fully open. This reduces blood flow from the heart to the aorta and thus to the rest of the body. With the advent of minimally invasive procedures, valve replacement may currently be the most common therapy for aortic valve stenosis. More particularly, transvenous/transcatheter aortic valve replacement (TAVR) is becoming a more common valve replacement procedure, as technology and doctor skillsets improve. This minimally invasive approach to valve replacement is an alternative to open heart surgical aortic valve replacement (SAVR). In a TAVR procedure, the damaged valve, whether the valve is a natural aortic valve or a previous SAVR or TAVR valve, is left in place. These valves may have anatomical abnormalities, calcification, or infection. TAVR has been approved for low-risk patients, who in general are of younger age and live longer. Recent studies have shown that the life of a TAVR valve will only be on average 8 years, so there will be an increase in replacement TAVR procedures. Old TAVR leaflets may be fibrosed, calcified, or thickened during the life of the TAVR valve, creating a barrier to the replacement TAVR valve. The old leaflets may also pose a more serious risk of coronary obstruction than native valves.

Thus, certain structural heart procedures such as trans-catheter valve replacement can cause the leaflets of the native valve, or those of a previously implanted valve, to be forced outward radially from the valve, thereby closing off either vessels that originate in the vicinity of the valve, or the blood path downstream from the valve (outflow tract).

Accordingly, many structural heart procedures, including TAVR and other valve replacement procedures, benefit from slitting of the leaflets of native and/or implanted valves. However, with current methods, there is no practical way to produce multiple slits in a heart valve leaflet, even where it is advantageous to do so. Thus, a need exists for improved intracoronary methods to address the foregoing and other concerns.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Disclosed herein is a multi-tined leaflet puncture and slitting device, with associated systems and methods. The device includes multiple first electrodes capable of puncturing the tissue of the leaflet and multiple second, hook-shaped electrodes capable of extending the puncture to slit the leaflet. The electrodes are energized with electrical energy. This leaflet puncture and slitting device enables a clinician to simultaneously produce multiple punctures the leaflet in precise locations and then simultaneously produce multiple slits in a controlled manner, in a direction away from the wall of the heart or aorta, thus preventing unintended damage to nearby anatomical structures. The leaflet puncture and slitting device has particular but not exclusive utility for modifying heart valve leaflets during intracardiac valve replacements.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an apparatus that includes a puncture and slitting device, which may include: a flexible elongate member configured to be positioned within a body lumen of a patient, and a cutting assembly positioned at a distal portion of the flexible elongate member. The cutting assembly may include: a first conductor, a crossing electrode configured to be energized by the first conductor, a second conductor electrically isolated from the first conductor, and a cutting electrode configured to be energized by the second conductor. Other examples of this aspect include corresponding computer systems, apparatus, and computer programs recorded on one or more computer storage devices, each configured to perform the actions of the methods.

Implementations may include one or more of the following features. In some aspects, the crossing electrode is cylindrical-shaped, conical-shaped, or wedge-shaped. In some aspects, the body lumen may include an aorta or a heart chamber of the patient, and the crossing electrode is configured to form a puncture in a heart valve leaflet when placed against the heart valve leaflet and energized by the first conductor. In some aspects, the cutting electrode may include a hook shape. In some aspects, the hook shape facilitates slitting of the heart valve leaflet when the cutting electrode is: placed within the puncture; energized by the second conductor; and pulled along a length of the heart valve leaflet. In some aspects, the second conductor may include a conductive tube, braid, or spring, where a first portion of the conductive tube, braid, or spring is surrounded by an outer insulation layer, where a second portion of the conductive tube, braid, or spring is not surrounded by the outer insulation layer, and where the second portion may include the cutting electrode. In some aspects, the first conductor may include a core wire, where the conductive tube, braid, or spring is positioned around the core wire and separated from the core wire by an inner insulation layer. In some aspects, at least one of the first conductor or the second conductor may include a shape memory material. In some aspects, the apparatus may include a power source configured to energize: the crossing electrode via the first conductor; and the cutting electrode via the second conductor. In some aspects, the first conductor may include a pullwire, where the cutting electrode is configured to transition from a straight configuration to a hook-shaped configuration when the pullwire is pulled. In some aspects, the second conductor may include a conductive hypotube, which may include a lumen, where the first conductor is positioned within the lumen and electrically isolated from the first conductor by an inner insulation layer surrounding the first conductor. In some aspects, the cutting electrode may include a cut portion of the conductive hypotube. In some aspects, the cut portion of the conductive hypotube may include gaps configured to provide flexibility for the cutting electrode to transition between a straight configuration and a hook-shaped configuration. In some aspects, the lumen of the hypotube is configured to receive a fluid, where the gaps in the cut portion are configured to allow the fluid to flow out of the puncture and slitting device and into the body lumen, thereby suppressing electrical conduction by blood proximate to the cutting electrode. In some aspects, the cutting electrode is covered by an insulator that may include a plurality of openings. In some aspects, a thickness of the insulator and a width of the plurality of openings are selected such that electrical energy emitted by the cutting electrode is configured to cut the tissue without direct contact between the cutting electrode and the tissue. In some aspects, the plurality of openings is rectangular, circular, or slotted. In some aspects, the plurality of openings is arranged in a regular pattern. Implementations of the described techniques may include hardware, a method or process, or computer software on a computer-accessible medium.

One general aspect includes an apparatus including a flexible, elongate intracardiac device that may include: a crossing electrode defining a distal end of the intracardiac device and configured to cut a hole in a heart valve leaflet; and a cutting electrode proximal of the crossing electrode and configured to cut a slit starting from the hole in the heart valve leaflet, where the cutting electrode may include a portion of a hypotube with gaps, where the gaps are configured to provide flexibility for the cutting electrode to transition between a straight configuration and a hooked shape configuration, where the crossing electrode is configured to cut the hole while the cutting electrode is in the straight configuration, and where the cutting electrode is configured to cut the slit while the cutting electrode is in the hook-shaped configuration. Other examples of this aspect include corresponding computer systems, apparatus, and computer programs recorded on one or more computer storage devices, each configured to perform the actions of the methods.

Implementations may include one or more of the following features. In some aspects, the intracardiac device further may include a pullwire conductor electrically coupled to the crossing electrode and electrically isolated from the cutting electrode, where the pullwire conductor is configured to carry electrical energy to power the crossing electrode to cut the hole, where the cutting electrode is configured transition from the straight configuration to the hook-shaped configuration when tension is applied to the pullwire conductor. Implementations of the described techniques may include hardware, a method or process, or computer software on a computer-accessible medium.

One general aspect includes an apparatus that includes a flexible elongate member configured to be positioned within a patient body and may include: a first tine that may include a first crossing electrode and a first cutting electrode; a second tine that may include a second crossing electrode and a second cutting electrode; and a polymer jacket positioned around the first tine and the second tine, where the polymer jacket is configured to prevent longitudinal motion and rotational motion of the first tine and the second tine relative to one another, where the first tine and the second tine are configured to transition between a straight configuration and a hook-shaped configuration, where the first crossing electrode and the second crossing electrode are configured to cut anatomy of the patient body while the first tine and the second tine are in the straight configuration, where the first cutting electrode and the second cutting electrode are configured to cut the anatomy while the first tine and the second tine are tine are in the hook-shaped configuration. Other embodiments of this aspect include corresponding computer systems, apparatus, and computer programs recorded on one or more computer storage devices, each configured to perform the actions of the methods.

Implementations may include one or more of the following features. In some aspects, the first tine and the second tine each may include a proximal portion and a distal portion, where the polymer jacket is positioned around the proximal portion of the first tine and the second tine. In some aspects, the first crossing electrode and the first cutting electrode are positioned at the distal portion of the first tine, where the second crossing electrode and the second cutting electrode are positioned at the distal portion of the second tine, where the polymer jacket is not positioned around the distal portion of the first tine and the second tine such that the distal portion of the first tine and the second tine are uncovered to allow transition between the straight configuration and the hook-shaped configuration. In some aspects, the flexible elongate member may include a third tine may include a third crossing electrode and a third cutting electrode. In some aspects, the first tine, the second tine, and the third tine are arranged in a triangular configuration. In some aspects, the jacket is configured to fixedly maintain the first tine, the second tine, and the third tine in the triangular configuration. In some aspects, the polymer jacket is bonded with a polymer of the first tine, a polymer of the second tine, and a polymer of the third tine. In some aspects, the first tine and the second tine are more radially spaced from one another in the hooked configuration than in the straight configuration. In some aspects, the second tine is configured to have radial motion in a first direction relative to the first tine when the first tine and the second tine transition from the straight configuration to the hooked configuration. In some aspects, the flexible elongate member may include a third tine configured to transition between the straight configuration and the hook-shaped configuration, where the third tine is configured to have radial motion in an opposite, second direction relative to the first tine when the first tine, the second tine, and the third tine transition from the straight configuration to the hook-shape configuration. In some aspects, the first tine may include a central tine, where the second tine is positioned on a first side of the central tine, and the third tine is positioned on an opposite second side of the central tine. In some aspects, the first tine and the second tine may include a different structure from one another. In some aspects, the second tine and not the first tine may include a first plurality of slits configured to provide radial motion relative to the first tine, where the plurality of slits are positioned proximal of the second crossing electrode and the second cutting electrode. In some aspects, the first tine and the second tine may include a second plurality of slits configured to provide deflection, in a same direction, for: the first crossing electrode and the first cutting electrode; and the second crossing electrode and the second cutting electrode. In some aspects, the anatomy may include a heart valve leaflet, where, to cut the heart valve leaflet, the first crossing electrode and the second crossing electrode are configured to simultaneously puncture the heart valve leaflet, where, to cut the heart valve leaflet, the first cutting electrode and the second cutting electrode are configured to simultaneously slit the heart valve leaflet. In some aspects, the first crossing electrode and the second crossing electrode are configured to simultaneously puncture the heart valve leaflet during an individual first movement of the intracardiac device, and where the first cutting electrode and the second cutting electrode are configured to simultaneously slit the heart valve leaflet during an individual second movement of the intracardiac device. In some aspects, the individual first movement of the intracardiac device may include a forward longitudinal movement of the intracardiac device while in a straight configuration, where the individual second movement of the intracardiac device may include a backward longitudinal movement of the intracardiac device while in a hook-shaped configuration. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A is a side view of a human heart according to aspects of the present disclosure.

FIG. 1B is a cross-sectional side view of a human heart according to aspects of the present disclosure.

FIG. 2 is a cross-sectional side view of an aortic valve replacement in a human heart 100 according to aspects of the present disclosure.

FIG. 3A is a diagrammatic, cross-sectional top view of an aortic valve replacement of a degenerated natural aortic valve utilizing a TAVR valve, according to aspects of the present disclosure.

FIG. 3B is a diagrammatic cross-sectional top view of a valve-in-valve replacement of a degenerated TAVR valve utilizing a new TAVR valve, according to aspects of the present disclosure.

FIG. 3C is a diagrammatic cross-sectional top view of a valve-in-valve replacement of a degenerated surgical aortic valve replacement (SAVR) valve utilizing a new TAVR valve, according to aspects of the present disclosure.

FIG. 4A is a diagrammatic top view of an aortic valve, according to aspects of the present disclosure.

FIG. 4B is a diagrammatic cross-sectional side view of a TAVR implant in an aortic valve according to aspects of the present disclosure.

FIG. 5 is schematic, diagrammatic view of a system according to aspects of the present disclosure.

FIG. 6A is a diagrammatic cross-sectional side view of an aortic valve according to aspects of the present disclosure.

FIG. 6B is a diagrammatic cross-sectional side views of an aortic valve according to aspects of the present disclosure.

FIG. 7A is a diagrammatic cross-sectional side view of an aortic valve according to aspects of the present disclosure.

FIG. 7B is a diagrammatic cross-sectional side view of an aortic valve according to aspects of the present disclosure.

FIG. 8 is a schematic, diagrammatic side view of an example resection wire of an example resection wire of a leaflet puncture and slitting device, according to aspects of the present disclosure.

FIG. 9 is a schematic, diagrammatic side view of an example resection wire of a leaflet puncture and slitting device, according to aspects of the present disclosure.

FIG. 10 is a schematic, diagrammatic, side cross-sectional view of at least a portion of an example leaflet puncture and slitting device, according to aspects of the present disclosure.

FIG. 11 is a schematic diagram of a processor circuit, according to aspects of the present disclosure.

FIG. 12 is a side view of a distal portion of an example resection wire or device in a straight configuration, according to aspects of the present disclosure.

FIG. 13 is a side cross-sectional view of the resection device of FIG. 26, according to aspects of the present disclosure.

FIG. 14A is a side view of the resection device of FIG. 26 in the hooked configuration, according to aspects of the present disclosure.

FIG. 14B is a side cross-sectional view of the resection device of FIG. 26 in the hooked configuration, according to aspects of the present disclosure.

FIG. 15 is a schematic, diagrammatic representation of a multi-tine tissue resection wire, according to aspects of the present disclosure.

FIG. 16 is a schematic, diagrammatic representation of a three-tine tissue resection wire in its straight configuration, according to aspects of the present disclosure.

FIG. 17 is a schematic, diagrammatic representation of a multi-tine tissue resection wire, having at least two tines, in their straight configuration, according to aspects of the present disclosure.

FIG. 18 is a schematic-diagrammatic end view of a three-tine tissue resection wire in its straight configuration, according to aspects of the present disclosure.

FIG. 19 is a schematic, diagrammatic perspective view of a three-tine tissue resection wire in its hooked configuration, according to aspects of the present disclosure.

FIG. 20 is a schematic, diagrammatic end view of an example three-tine resection wire in its hooked configuration, according to aspects of the present disclosure.

FIG. 21 is a schematic, diagrammatic top view of an example three-tine resection wire in its hooked configuration, according to aspects of the present disclosure.

FIG. 22A is a diagrammatic top view of an aortic valve, according to aspects of the present disclosure.

FIG. 22B is a diagrammatic top view of an aortic valve, according to aspects of the present disclosure.

### DETAILED DESCRIPTION

Disclosed herein is a leaflet puncture and slitting device, with associated systems and methods. During a heart valve replacement or other intracardiac procedure, slitting a heart valve leaflet from the center of the valve annulus towards the root of the leaflet (or vice versa) can allow for blood flow and/or passage of a guidewire. Procedures incorporating such a slitting process may be known as BASILICA or LAMPOON procedures, depending upon which heart valve is being repaired or replaced. This procedure uses a guidewire to pierce the leaflet near the root, and then the guide wire tip is snared, and externalized. Once both ends of the wire are under direct physician control (outside of the patient body), radio frequency energy is applied to the wire, which allows it to “cut” through the leaflet as tension is applied to the ends of the wire. This creates a slit in the leaflet from the root towards the center of the annular space.

These procedures may employ a normal guide wire, with a short section of the coating on the wire, near the middle of the wire, being scraped off and heated with RF energy for use as a cutting wire. When the uncoated area of the wire is in contact with the leaflet, the wire is connected to a radio frequency (RF) generator. When energy is applied, the wire can cut through valve leaflet. An advantage of this method is that wire will self-navigate around calcific tissue that can occur in a leaflet. Another advantage of this method is that it is relatively inexpensive, requiring only a standard wire and standard RF generator.

However, the BASILICA and LAMPOON procedures can be very technically challenging, requiring very advanced wire technique to both pierce the leaflet, especially in the area intended, and also to snare the tip of the wire to externalize the distal end. It may also be difficult to know when the uncoated section of wire is in the correct position to slit the valve leaflet. Furthermore, if the uncoated section is made too large, it could damage tissue that is not intended to be resected. With current systems, there is also no practical way to produce multiple slits in the valve leaflet.

To address these and other problems, disclosed herein is a multi-tined leaflet puncture and slitting device, with associated systems and methods. The device includes multiple first heated electrodes capable of puncturing the tissue of the leaflet, and multiple second, hook-shaped heated electrodes capable of extending the puncture into a slit, from the base of the leaflet to its tip. This multi-tined leaflet puncture and slitting device enables a clinician to simultaneously produce multiple punctures the leaflet in precise locations and then simultaneously produce multiple slits in the leaflet in a controlled manner, in a direction away from the wall of the heart or aorta, while reducing the risk of damage to nearby structures.

The leaflet puncture and slitting device facilitates the piercing of the leaflet by using RF energy to easily allow the wire to puncture the leaflet tissue, and prevents the need to snare the wire and externalize it by using a wire that is pre-shaped into a “hook” configuration.

The leaflet puncture and slitting device includes a resection wire that branches into multiple hook shapes, each made from a super-elastic alloy material with at least two separate electrodes. The resection wire may be coated on the surface with an insulative coating, except on the electrodes themselves. The leaflet puncture and slitting device also includes a means to easily connect the resection wire to an RF generator, such as an industry standard connector.

The device can be constructed in a number of ways. For example, it can be made from a hollow conductive tube which would form the body of the wire, the hook, and one or more of at least two electrodes – a cutting electrode. The hollow tube can also form one of two necessary electrical connections to a radio frequency (RF) generator (e.g., the connection to the first electrodes or cutting electrodes). Inside the hollow tube, there may be an insulation layer around a wire which forms a second electrode at the tips of each tine – the crossing electrodes. This resection wire can also become the connection point for the second electrodes to the RF generator.

Examples of related, single-tined leaflet resection devices can be found in U.S. Provisional Application No. 63/527,871, filed July 20, 2023, entitled “Multiple Electrode Heart Valve Slitting Device” and International Application No. PCT/EP2024/069683, filed July 11, 2024, entitled “Multiple Electrode Heart Valve Slitting Device”, each of which is incorporated by reference as though fully set forth herein.

In an example, the resection wire is a first flexible elongate member that fits within a second flexible elongate member (e.g., a guide catheter). In use, the catheter is introduced into the vasculature of the patient and directed to the valve leaflet of interest. Once there, the user extends the resection wire distally from the catheter until the crossing electrodes are in contact with the leaflet. Radio frequency (RF) energy (e.g., from a standard electrocautery source) is applied to the crossing electrodes in order to puncture the leaflet. Then the RF energy is switched off, and the resection wire is advanced through the puncture until the cutting electrodes are within the puncture. Then, RF energy is applied to the cutting electrodes, and the resection wire is pulled backward into the catheter, which causes the heated cutting electrode to produce multiple slits in the leaflet.

The present disclosure aids substantially in intracardiac procedures such as heart valve replacement, by improving a clinician’s ability to cut (e.g., resect) a valve leaflet with precision, and with minimal risk of damage to neighboring tissues. Implemented on a guidewire-like assembly deployed from an intracardiac catheter in association with an intracardiac catheter system, the multi-tined leaflet puncture and slitting device disclosed herein provides practical, precise surgical capabilities in an intracardiac environment. This improved leaflet modification technique transforms a complex, potentially imprecise procedure requiring high levels of skill and training into a repeatably precise leaflet slitting technique, without the normally routine need to achieve this precision through open-heart surgical interventions. This unconventional approach improves the functioning of the intracardiac catheter system, by allowing a greater range of procedures to be reliably performed using intracardiac techniques, and by allowing multiple slits to be produced in the valve leaflet.

Control of the leaflet puncture and slitting device may be implemented at least partially through ultrasound or X-ray imaging under the control of a processor and viewable on a display, and operated by a control process executing on a processor that accepts user inputs from a keyboard, mouse, or touchscreen interface. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

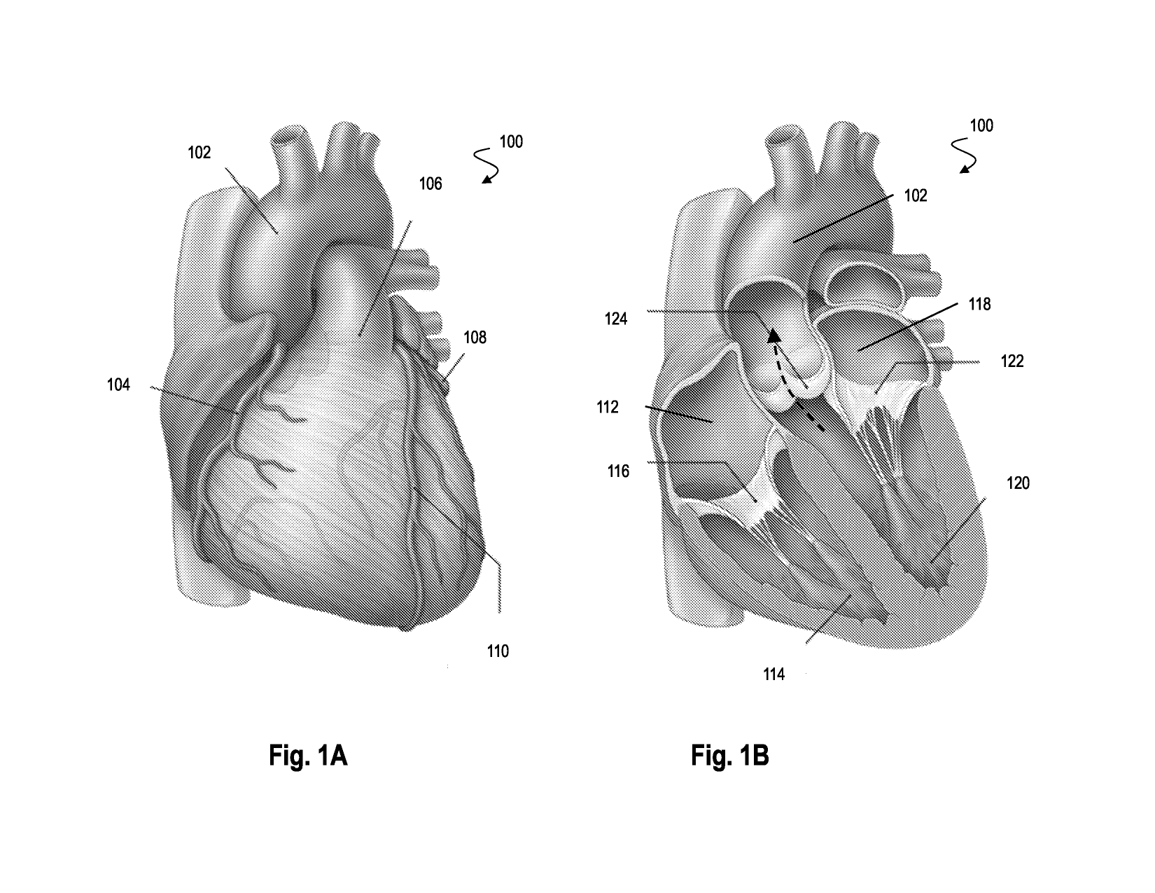

FIG. 1A is a side view of a human heart 100 according to aspects of the present disclosure. Visible are an aorta 102 from which stems a right coronary artery 104 and a left main coronary artery 106. The left main coronary artery 106 branches into a left circumflex coronary artery 108 and a left anterior descending coronary artery 110. The right coronary artery 104, the left main coronary artery 106, the left circumflex coronary artery 108, and a left anterior descending coronary artery 110 are the arteries that provide oxygen-rich blood to muscles of the human heart 100.

FIG. 1B is a cross-sectional side view of a human heart 100 according to aspects of the present disclosure. Visible are a right atrium 112 and a right ventricle 114. In that regard, oxygen-poor blood enters the human heart 100 in the right atrium 112 and travels to the right ventricle 114 through the tricuspid valve 116. The oxygen-poor blood leaves the right ventricle 114 and travels to the lungs. Also visible are a left atrium 118 and a left ventricle 120. In that regard, oxygen-rich blood is received from the lungs in the left atrium 118 and travels to the left ventricle 120 through the mitral valve 122. The oxygen-rich blood leaves the left ventricle 120 and goes out to the body through the aorta 102 via an aortic valve 124.

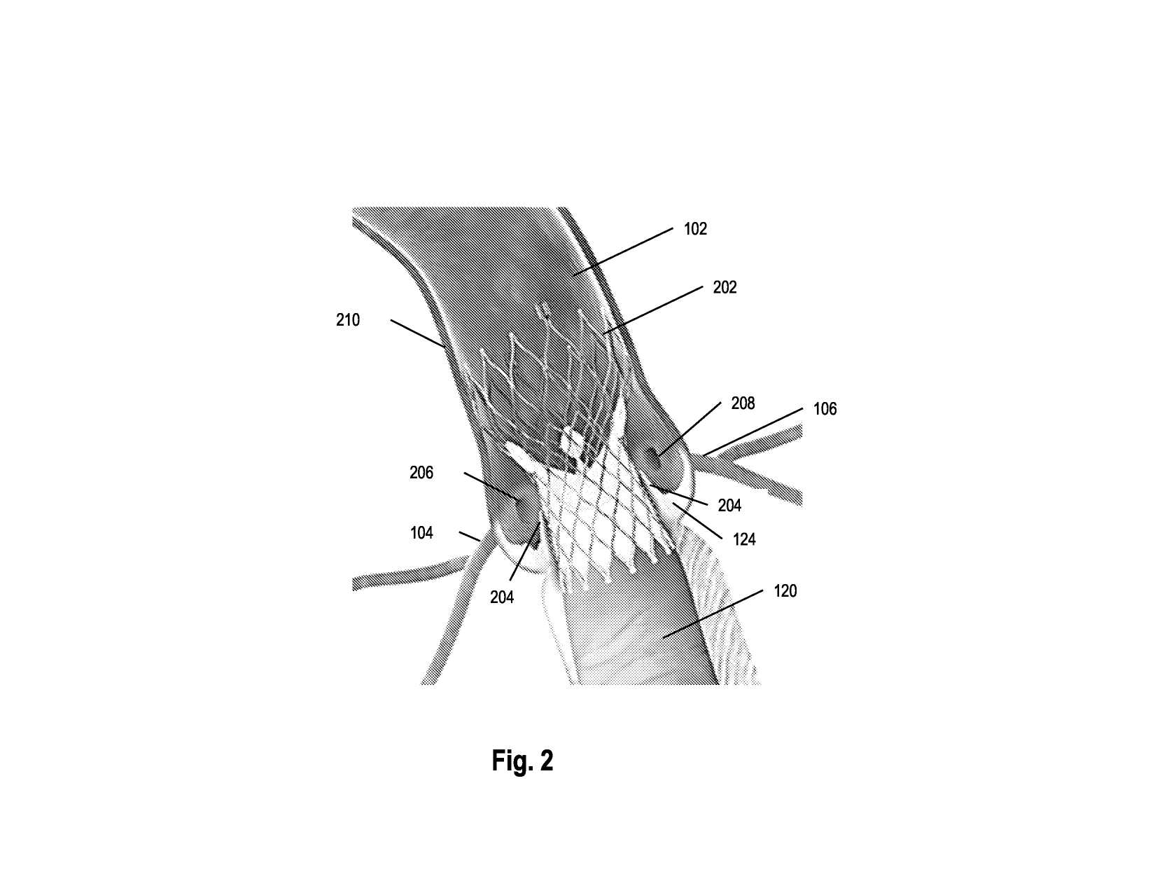

FIG. 2 is a cross-sectional side view of an aortic valve 124 replacement in a human heart 100 according to aspects of the present disclosure. In some aspects, e.g., when aortic valve stenosis has occurred to the aortic valve 124 that keeps blood flowing in the correct direction from the left ventricle 120 to the aorta 102, a transvenous/transcatheter aortic valve repair (TAVR) procedure may be performed to replace the natural aortic valve 124 with a replacement aortic valve 202. In some instances, portions of one or more leaflets 204 of the natural aortic valve 124 may be resected so that openings 206 and 208 to the right coronary artery 104 and the left main coronary artery 106, respectively, so that, when the one or more leaflets 204 are pressed against the natural heart wall 210, the openings 206 and 208 remain unobstructed so that oxygen-rich blood may flow to the muscles of the human heart. The natural heart wall 210 may be a natural aorta wall, a natural heart chamber wall, or a natural aortic valve wall.

The TAVR procedure is shown here for exemplary purposes only; it is understood that other heart valves and heart valve replacement procedure types may benefit from leaflet modification and thus fall within the scope of the present disclosure.

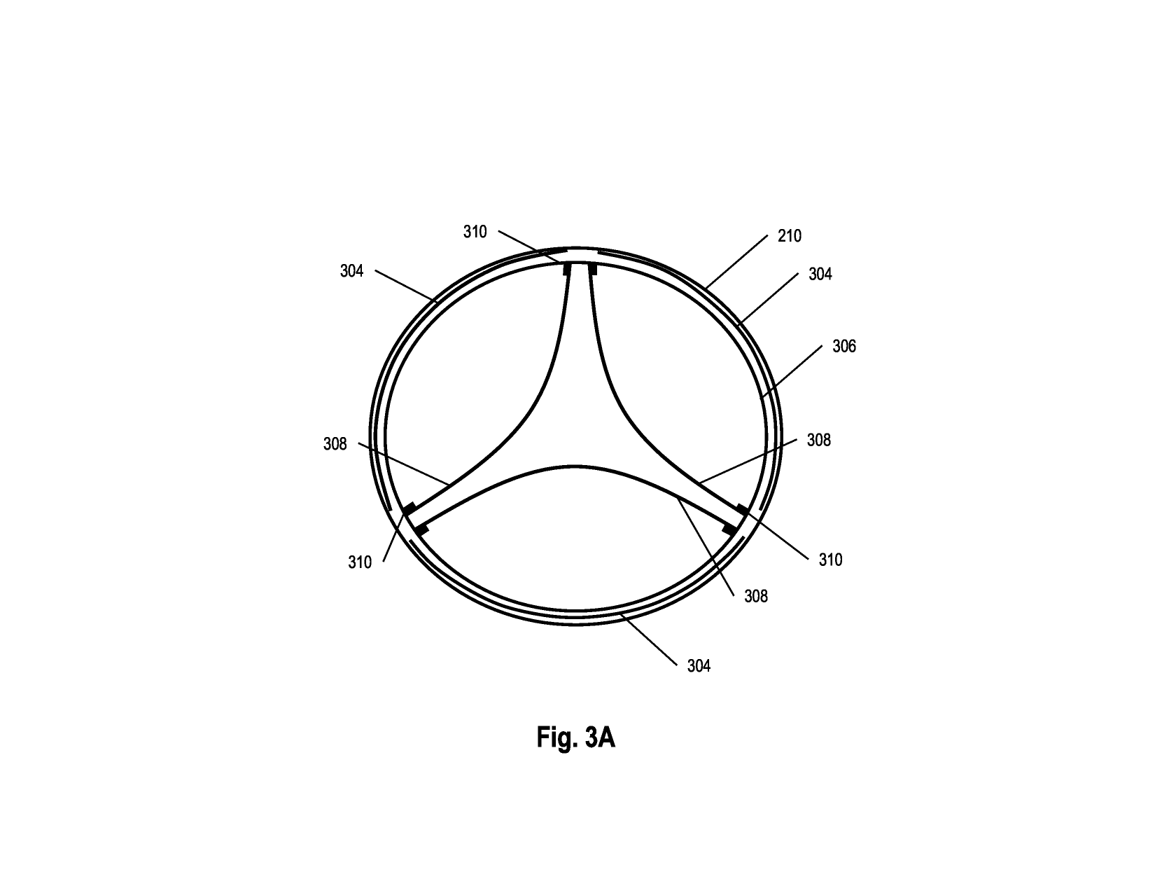

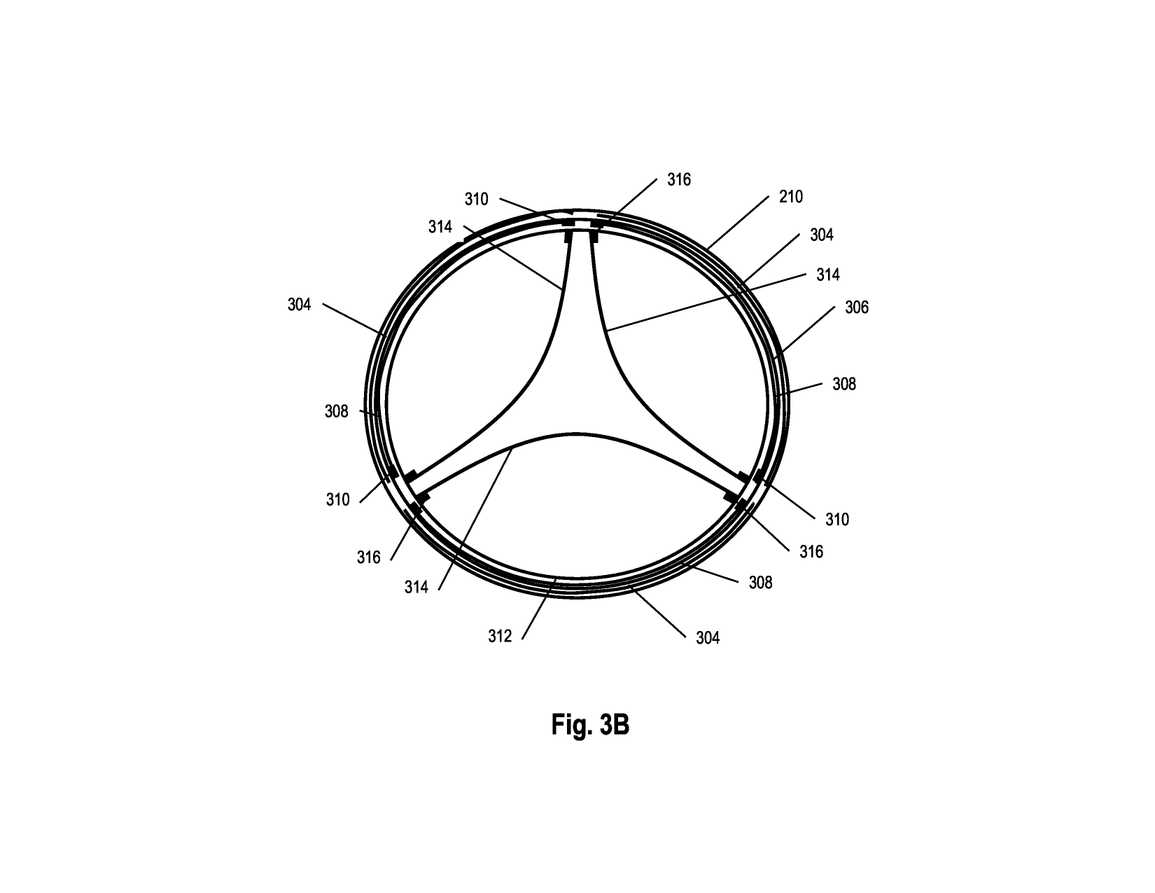

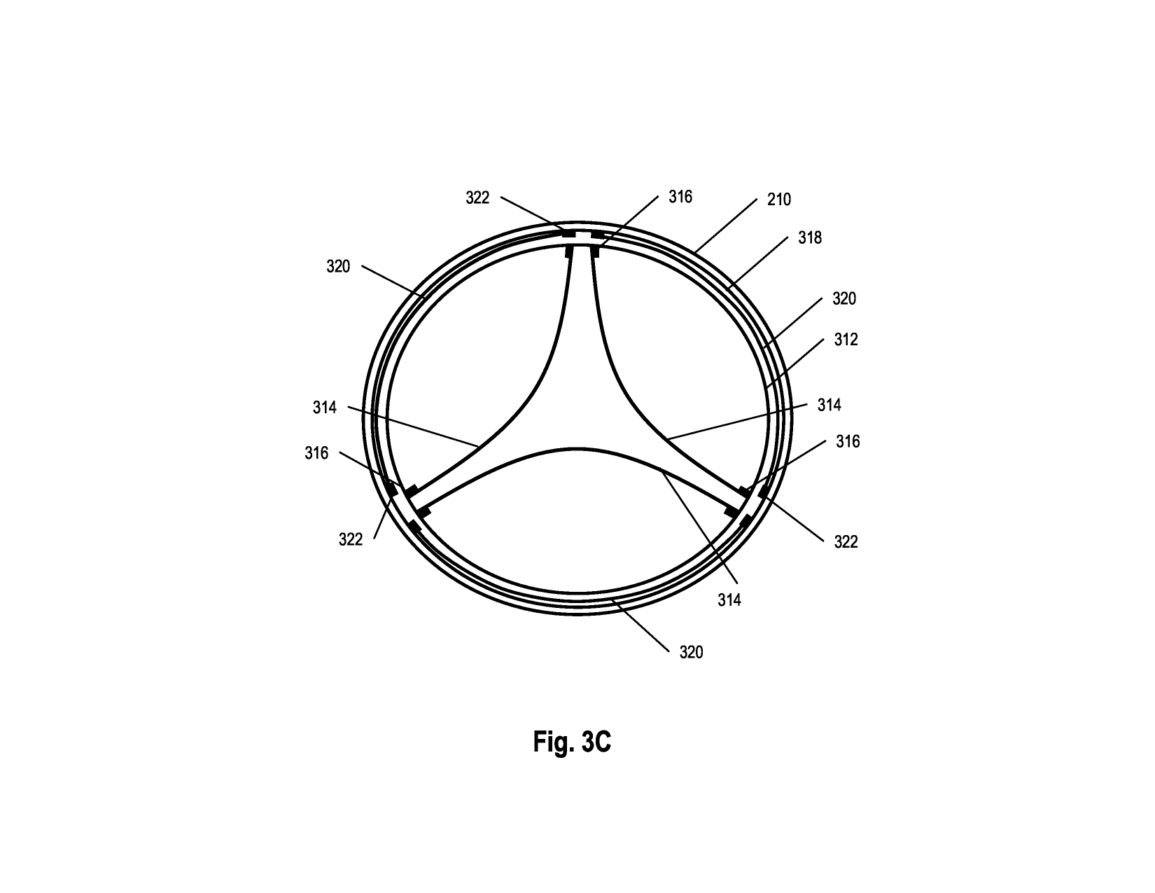

FIG. 3A is a diagrammatic, cross-sectional top view of an aortic valve replacement of a degenerated natural aortic valve utilizing a TAVR valve, according to aspects of the present disclosure. Visible are the natural heart wall 210, the natural aortic valve leaflets 304, the TAVR valve wall 306, the TAVR valve leaflets 308, and the TAVR commissural tabs 310. The TAVR valve leaflets 308 may for example be constructed from bovine animal tissue coupled to the wire frame of the TAVR valve wall 306 via the commissural tabs 310. When inserted, the TAVR valve pushes the remaining unresected portions of the natural aortic valve leaflets 304 against the natural heart wall 210 such that the natural aortic valve leaflets 304 are pinned and/or secured between an outside of the TAVR valve wall 306 and the natural heart wall 210.

FIG. 3B is a diagrammatic cross-sectional top view of a valve-in-valve replacement of a degenerated TAVR valve utilizing a new TAVR valve, according to aspects of the present disclosure. Visible are the natural heart 210, the natural aortic valve leaflets 304, the degenerated TAVR valve wall 306, the degenerated TAVR valve leaflets 308, the degenerated TAVR commissural tabs 310, the new TAVR valve wall 312, the new TAVR valve leaflets 314, and the new TAVR commissural tabs 316. The new TAVR valve leaflets 314 are constructed from bovine animal tissue are coupled to the wire frame of the new TAVR valve wall 312 via the commissural tabs 316. When inserted, the TAVR valve pushes the remaining unresected portions of the degenerated TAVR valve leaflets 308 against an inside of the degenerated TAVR valve wall 306 such that the degenerated TAVR valve leaflets 308 are pinned and/or secured between an outside of the new TAVR valve wall 312 and the inside wall of the degenerated TAVR valve wall 306.

FIG. 3C is a diagrammatic cross-sectional top view of a valve-in-valve replacement of a degenerated surgical aortic valve replacement (SAVR) valve utilizing a new TAVR valve, according to aspects of the present disclosure. Visible are the natural heart wall 210, a degenerated SAVR valve wall 318, the degenerated SAVR valve leaflets 320, the degenerated SAVR commissural tabs 322, the new TAVR valve wall 312, the new TAVR valve leaflets 314, and the new TAVR commissural tabs 316. The new TAVR valve leaflets 314 are constructed from bovine animal tissue are coupled to the wire frame of the new TAVR valve wall 312 via the commissural tabs 316. When inserted, the TAVR valve pushes the remaining unresected portions of the degenerated SAVR valve leaflets 320 against an inside of the degenerated SAVR valve wall 318 such that the degenerated TAVR valve leaflets 320 are pinned and/or secured between an outside of the new TAVR valve wall 312 and the inside wall of the degenerated SAVR valve wall 318.

The natural valve, TAVR valve, and/or SAVR valve can be degenerated (e.g., not fully or correctly functioning) as a result of various causes. For example, the natural valve, TAVR valve, and/or SAVR valve can be degenerated because it is stenosed, because it is regurgitant, etc.

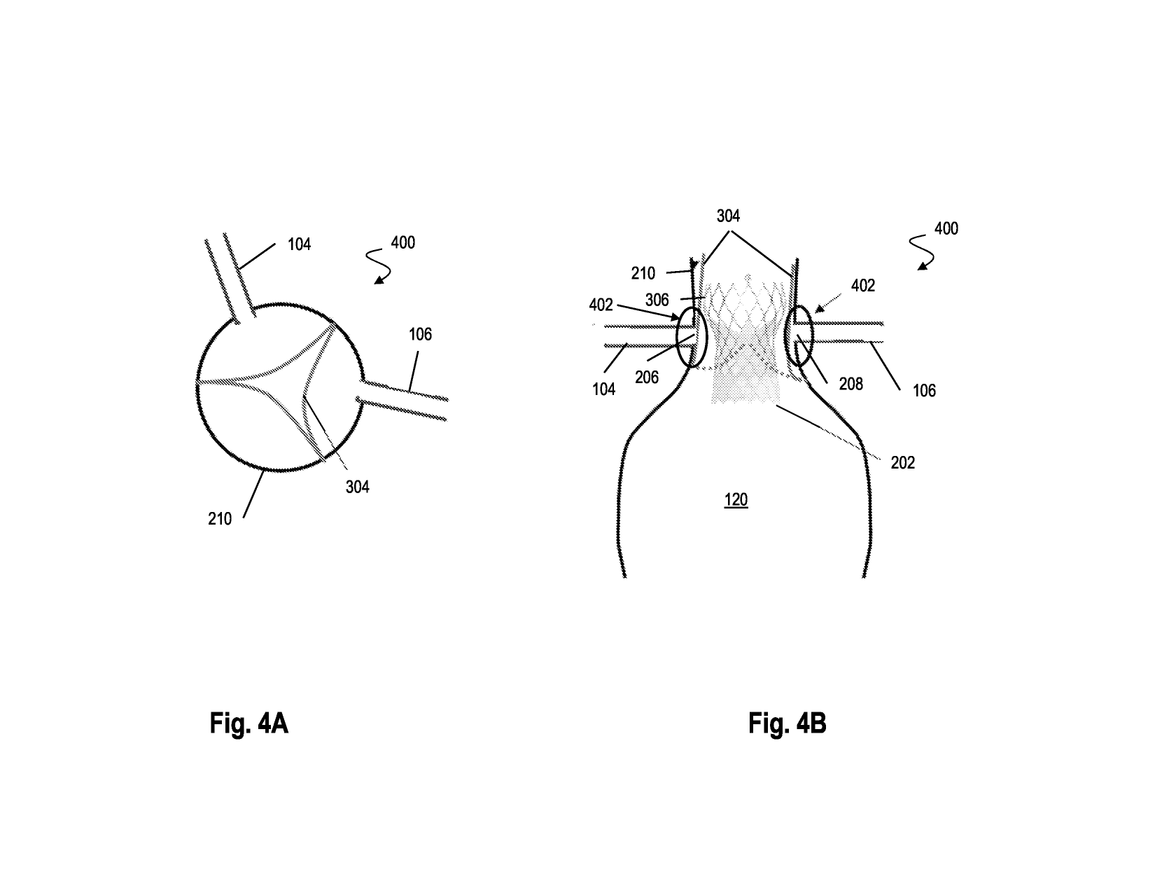

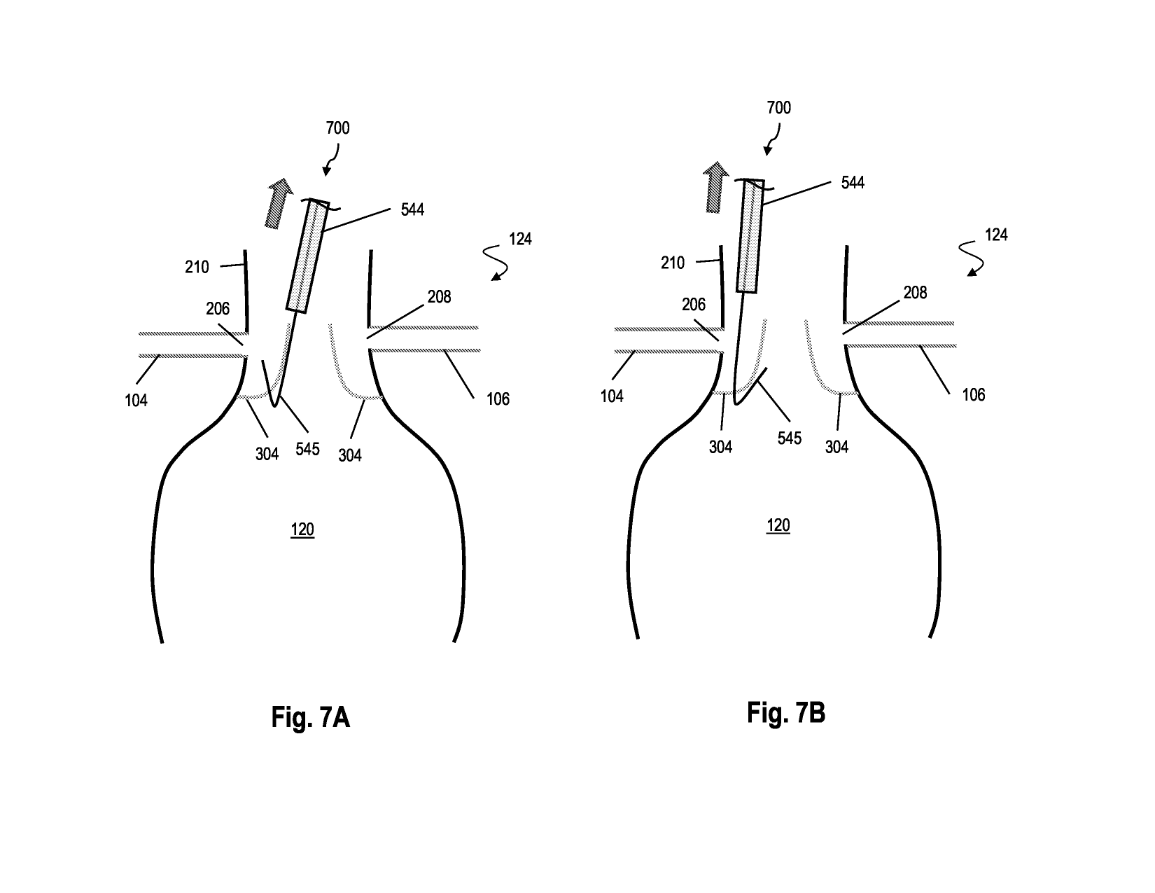

FIG. 4A is a diagrammatic top view of an aortic valve 400, according to aspects of the present disclosure. Visible are the natural heart wall 210, the aortic valve leaflets 304, the right coronary artery 104, and the left main coronary artery 106. During and after valve replacement procedures, the leaflets 304 can obstruct the arteries 104, 106, as described below in FIG. 4A. This can be alleviated by resecting one or more leaflets 304, as shown below in FIGS. 7 and 15-18B.

FIG. 4B is a diagrammatic cross-sectional side view of a TAVR implant in an aortic valve 400 according to aspects of the present disclosure. Visible are the natural heart wall 210, the aortic valve leaflets 304, the right coronary artery 104, and the left main coronary artery 106. In some aspects, when a replacement aortic valve 202 is implanted and the aortic valve leaflets 304 are not resected or insufficiently resected, the aortic valve leaflets 304, when pinned and/or secured between an outside of the TAVR valve wall 306 and the natural heart wall 210, obstruct the openings 206 and 208 to the right coronary artery 104 and the left main coronary artery 106, respectively, as indicated by areas 402.

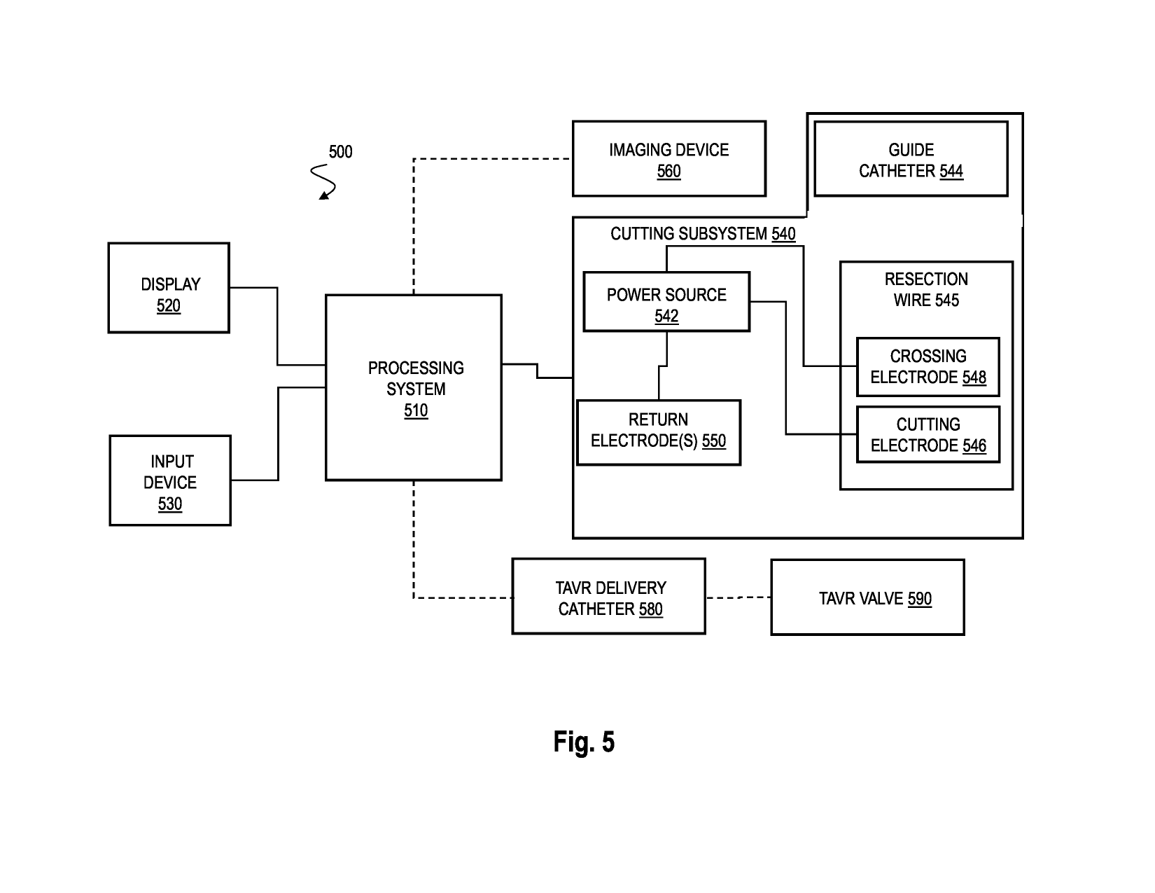

FIG. 5 is schematic, diagrammatic view of a system 500 according to aspects of the present disclosure. The system 500 may be configured to evaluate (e.g., assess), display, and/or control (e.g., modify) one or more aspects of a cardiac valve replacement. For instance, the system 500 may be utilized to monitor and/or control one or more portions of a cardiac valve replacement process while reducing and/or eliminating aortic regurgitation, as described in greater detail below. In this regard, the system 500 may be used to assess coronary vessels and/or heart tissue (e.g., the myocardium). As illustrated, the system 500 may include a processing system 510 in communication with a display device 520 (e.g., an electronic display or monitor), an input device 530 (e.g., a user input device, such as a keyboard, mouse, joystick, microphone, and/or other controller or input device), a cutting assembly or cutting subsystem 540 and/or an imaging device 560. As illustrated, the system 500 may further include a TAVR delivery catheter 580 and a TAVR valve 590, which may be completely mechanical or may be connected to processing system 510 in order to provide intraluminal data to processing system 510. It is understood that other types of valves and valve delivery systems may be used instead of or in addition to TAVR.

The cutting subsystem 540 includes a guide catheter 544, along with a power source 542 coupled to a resection wire 545, coupled to a puncturing element or crossing electrode 548 and cutting element or cutting electrode 546. The resection wire 545 can also be referred to as a resection catheter, resection device, etc., and/or combinations thereof. The power source 542 may for example be a generator of radio-frequency (RF) alternating current. The resection wire 545, including the crossing electrode 548 and cutting electrode 546, is delivered by the guide catheter 544 to the site of the leaflet of interest. Once properly located, the resection wire 545 may be extended from a distal end of the catheter 544, such that the crossing electrode 548 can puncture the leaflet and the cutting electrode 546 can resect the leaflet, as described below. In some cases, the cutting subsystem 540 may require a return electrode 550 in contact with the outside of the patient’s body. In other instances, the resection wire 545 may be a loop that includes both a source and return.

The processing system 510 is generally representative of any device suitable for performing the processing and analysis techniques disclosed herein. In some aspects, the processing system 510 includes a processor circuit, such as the processor circuit 2400 of FIG. 24. In some aspects, the processing system 510 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processing system 510 (e.g., computing device) using corresponding instructions stored on or in a non-transitory computer readable medium accessible by the computing device. In some instances, the processing system 510 is a console device. Further, it is understood that in some instances the processing system 510 includes one or a plurality of computing devices, such as computers, with one or a plurality of processor circuits. In this regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

The system 500 is configured such that when the resection wire 545 is positioned within the heart, images captured by the imaging system 560 can show the location and orientation of the resection wire 545, and also potentially anatomical features such as heart valve leaflets.

It is noted that block diagrams are provided herein for exemplary purposes; a person of ordinary skill in the art will recognize myriad variations that nonetheless fall within the scope of the present disclosure. For example, block diagrams may show a particular arrangement of components, subcomponents, modules, units, etc. It is understood that some embodiments of the systems disclosed herein may include additional components, that some components shown may be absent from some embodiments, and that the arrangement of components may be different than shown, while still performing the methods described herein.

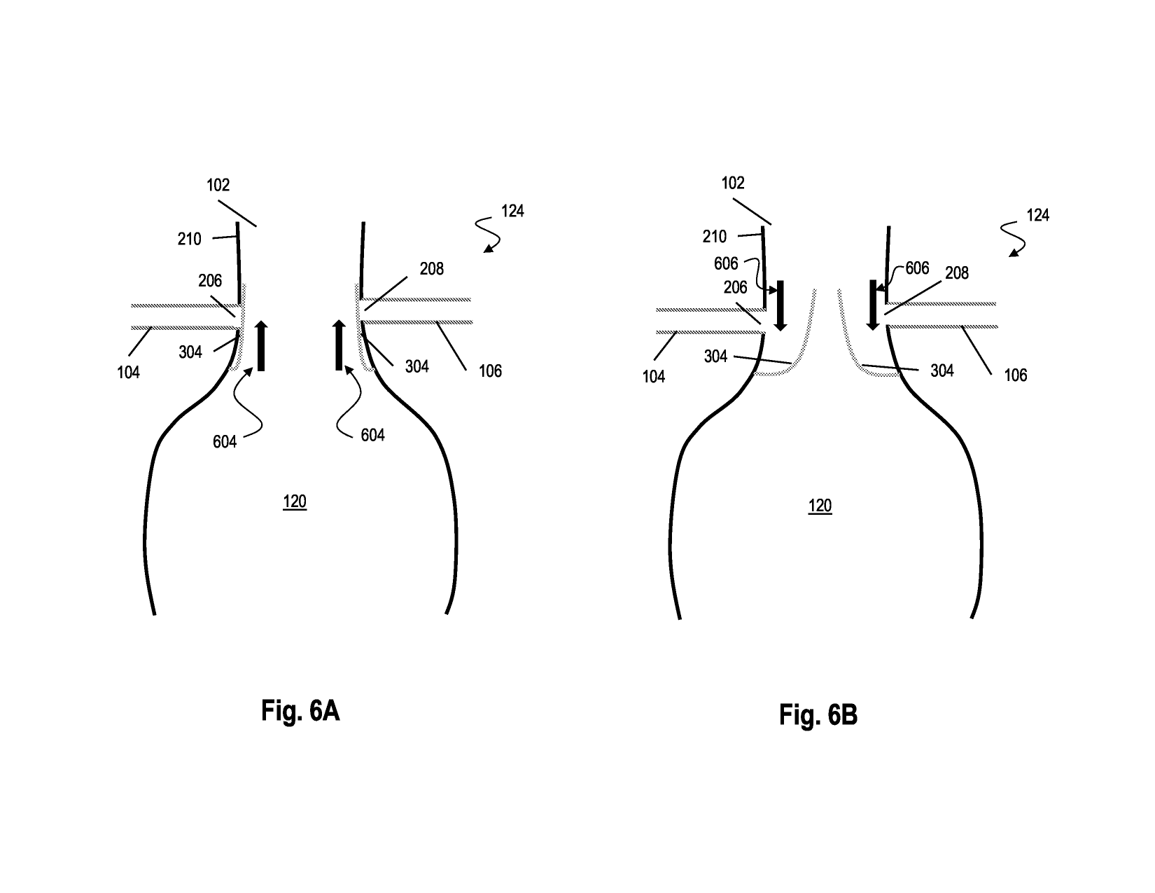

FIG. 6A is a diagrammatic cross-sectional side view of an aortic valve 124 according to aspects of the present disclosure. Visible are the natural heart wall 210, the aortic valve leaflets 304, the right coronary artery 104, and the left main coronary artery 106. In the aspects of FIG. 6A, during systole (indicated by directional arrows 604), the aortic valve leaflets 304 open and allow blood to flow from the left ventricle 120 to the aorta 102 indicated by directional arrows 604.

FIG. 6B is a diagrammatic cross-sectional side views of an aortic valve 124 according to aspects of the present disclosure. Visible are the natural heart wall 210, the aortic valve leaflets 304, the right coronary artery 104, and the left main coronary artery 106. In the aspects of FIG. 6B, during diastole (indicated by directional arrows 606), the aortic valve leaflets 304 may be pushed by the flow of blood from the aorta 102 toward the left ventricle 120 such that the aortic valve leaflets 304 close to prevent mitral regurgitation.

FIG. 7A is a diagrammatic cross-sectional side view of an aortic valve 124 according to aspects of the present disclosure. In the example shown in FIG. 7A, a leaflet puncture and slitting device 700 is being positioned onto one of the leaflets 304, in order to puncture and slit the leaflet as described below. The leaflet puncture and slitting device 700 advantageously enables the replacement of an aortic valve 124 in a non-obstructive way, by resecting old valve leaflets 304 and preparing the implant site for a cleaner valve deployment and operation. For example, resecting portions of the leaflets 304 of the degenerated aortic valve can allow the replacement valve to be expanded without the current leaflets 304 covering the openings to the coronary arteries 104, 106, thereby allowing oxygenated blood to flow to the muscles of the heart. The leaflet puncture and slitting device 700 includes a guide catheter 544 which has deployed a hook-shaped resection wire 545 that has punctured the leaflet 304.

FIG. 7B is a diagrammatic cross-sectional side view of an aortic valve 124 according to aspects of the present disclosure. In the example shown in FIG. 7B, the leaflet puncture and slitting device 700 is being positioned onto one of the leaflets 304, in order to puncture and slit the leaflet as described below. However, the orientation of the leaflet puncture and slitting device 700 is flipped relative to what is what is shown in FIG. 7A. More specifically, the resection wire 545 is puncturing the leaflet in a radially inward direction with respect to the aorta 102, rather than a radially outward direction. In either case, once the leaflet has been punctured, the leaflet can then be resected (e.g., slit) by pulling the resection wire into the guide catheter 544 (shown as an upward direction in FIGS. 7A and 7B), as described below.

Although FIGS. 7A and 7B illustrate the leaflet puncture and slitting device 700 being used on the aortic valve 124, it is also noted that that the device can be used to resect a leaflet of any heart valve (e.g., an aortic valve, mitral valve, pulmonary valve, or tricuspid valve). Furthermore, the leaflet puncture and slitting device 700 could be in the heart (e.g., a heart chamber) or a great vessel (e.g., the aorta) while it is gripping and slitting the leaflet.

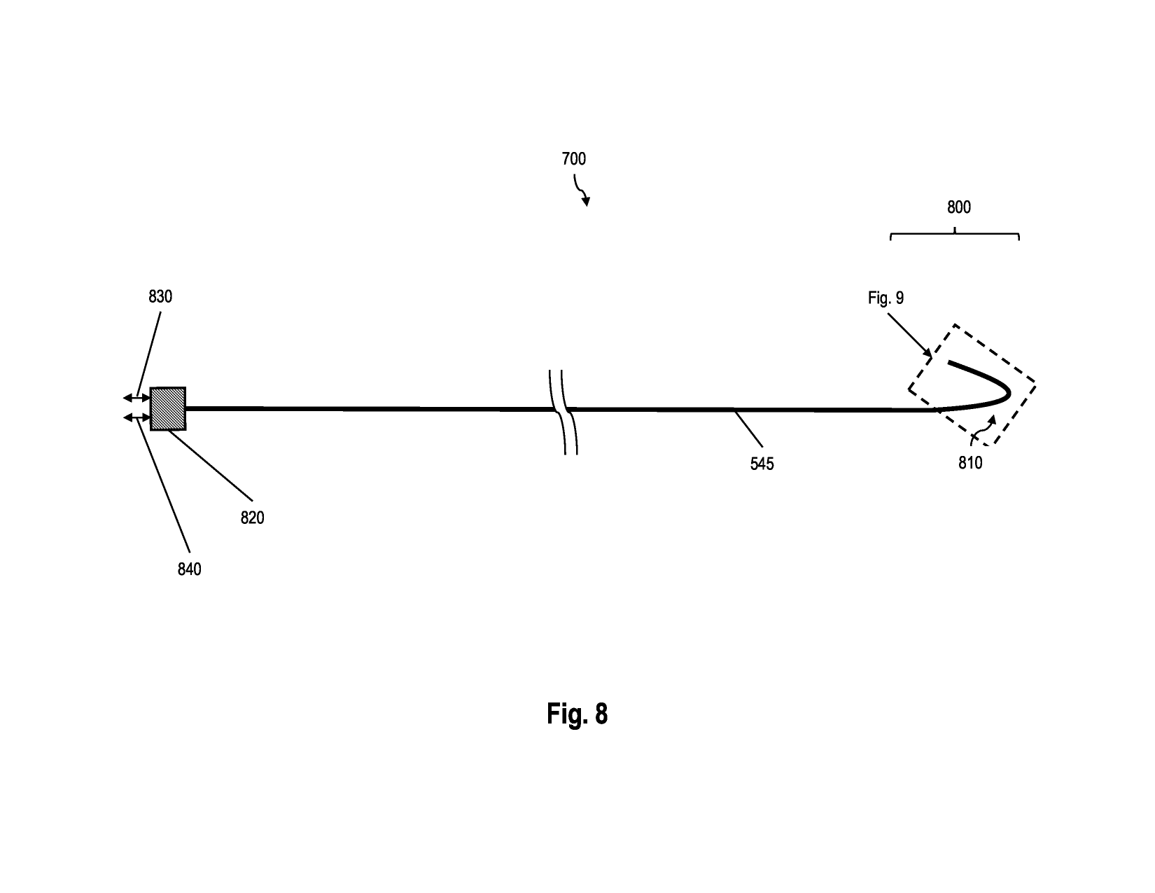

FIG. 8 is a schematic, diagrammatic side view of an example resection wire 545 of a leaflet puncture and slitting device 700, according to aspects of the present disclosure. The resection wire 545 includes a connector 820 to connect the resection wire 545 to a power source such as an RF generator (e.g., power source 542 of FIG. 5). The connector 820 includes a conductive portion 830 for carrying electrical signals and/or electrical energy to the crossing electrode 548 (see FIG. 5), and a conductive portion 840 for carrying electrical signals and/or electrical energy to the cutting electrode 546.

A distal portion 800 of the resection wire 545 includes a hook shape 810 to facilitate puncturing and/or slitting of the heart valve leaflet, as described below. The resection wire 545 may be a flexible elongate member sized, shaped, structured, and/or otherwise configured to be extend through blood vessels and into/proximate to major vessels of the heart and/or chambers of the heart. For example, the resection wire 545 can be sufficiently flexible to travel through blood vessels. However, the resection wire 545 may also have a sufficient degree of rigidity so that it can be pushed through blood vessels without kinking, and such that the hook shape 810 can remain hooked while resecting the leaflet.

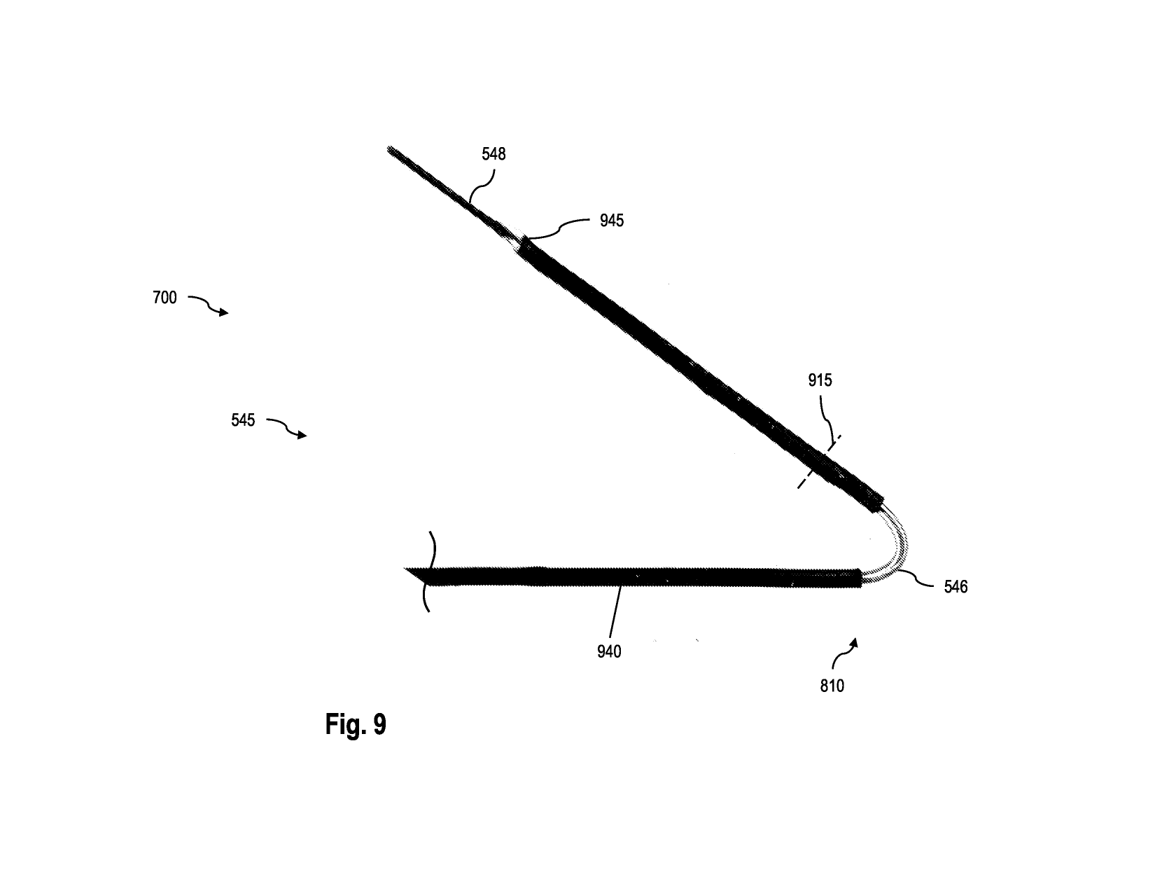

FIG. 9 is a schematic, diagrammatic side view of an example resection wire 545 of a leaflet puncture and slitting device 700, according to aspects of the present disclosure. Portions of the resection wire 545 are surrounded by an outer insulation layer 940 (e.g., heat shrink tubing). The crossing electrode 548 extends from the distal end of the resection wire 545, and may for example be a portion of a core wire extending beyond the distal end 945 of the outer insulation layer 940. When energized with RF electrical energy, the crossing electrode is capable of puncturing tissue such as a heart valve leaflet. The core wire may extend distally to the connector 820 (see FIG. 8).

In the example shown in FIG. 9, the hook-shaped portion 810 of the resection wire 545 includes the cutting electrode 546, which may for example be a metallic tube (e.g., a hypotube) surrounding the core wire, from which the outer insulation layer 940 has been partially stripped. When energized with RF electrical energy, the cutting electrode is capable of cutting tissue such as the heart valve leaflet. In an example, the metal tube may extend proximally all the way to the connector 820 (see FIG. 8), but may extend distally only to a location 915, between the cutting electrode 546 and the distal end 945 of the outer insulation layer 940.

Because the crossing electrode 548 and the cutting electrode 546 can be activated separately, the leaflet puncture and slitting device 700 provides the user (e.g., a clinician such as a heart surgeon) with precise control over where and how the RF energy is applied to the tissue, as described below.

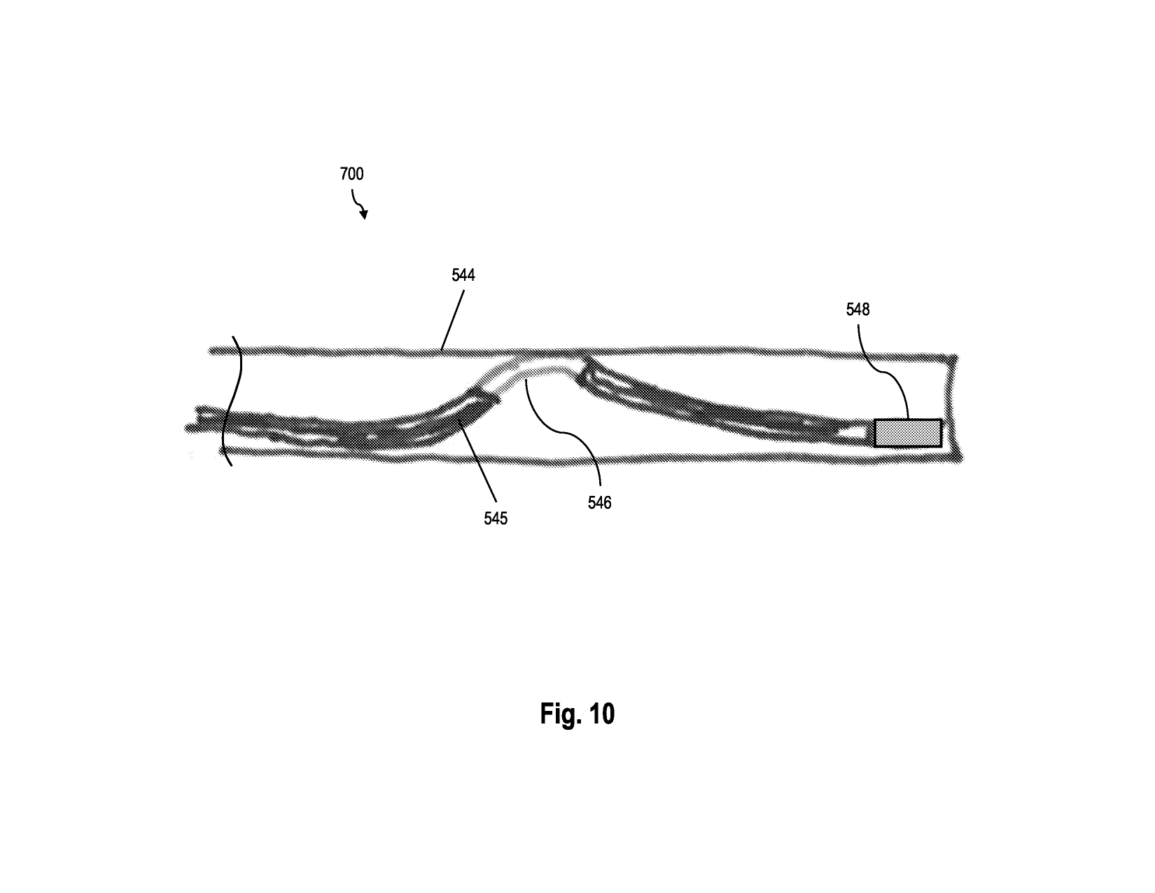

FIG. 10 is a schematic, diagrammatic, side cross-sectional view of at least a portion of an example leaflet puncture and slitting device 700, according to aspects of the present disclosure. The leaflet puncture and slitting device 700 can also include the resection wire 545 and the guide catheter 544 in some aspects. Prior to deployment, the resection wire 545 is positioned within the guide catheter 544 such that the cutting electrode 546 and the crossing electrode 548 are not exposed, and thus pose little risk of accidental tissue damage.

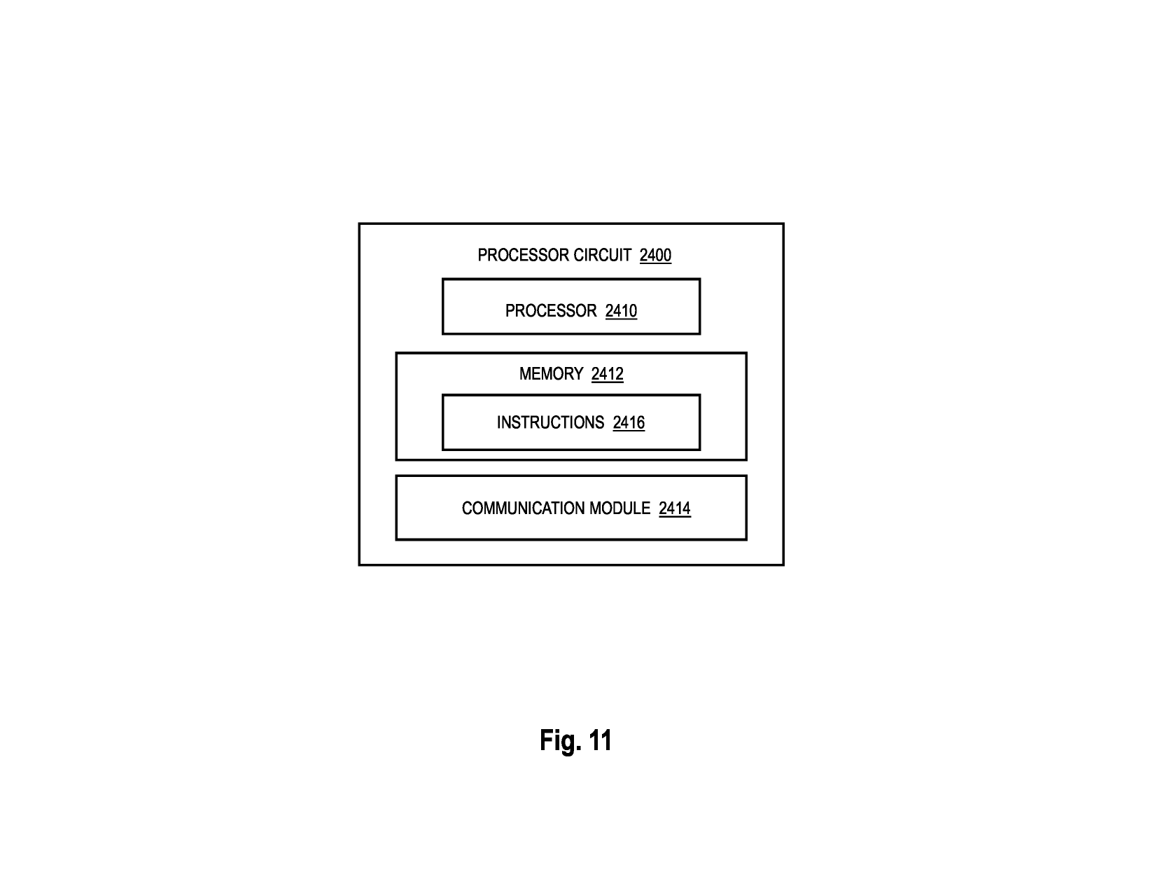

FIG. 11 is a schematic diagram of a processor circuit 2450, according to aspects of the present disclosure. The processor circuit 2450 may be implemented in the processing system 510, the system 500, or other devices or workstations (e.g., third-party workstations, network routers, etc.), or on a cloud processor or other remote processing unit, as necessary to implement the method. As shown, the processor circuit 2450 may include a processor 2460, a memory 2464, and a communication module 2468. These elements may be in direct or indirect communication with each other, for example via one or more buses.

The processor 2460 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 2460 may also comprise another hardware device, a firmware device, or any combination thereof configured to perform the operations described herein. The processor 2460 may also be implemented as a combination of computing devices, e.g., a combination of a DSP and a microprocessor, a plurality of microprocessors, one or more microprocessors in conjunction with a DSP core, or any other such configuration.

The memory 2464 may include a cache memory (e.g., a cache memory of the processor 2460), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 2464 includes a non-transitory computer-readable medium. The memory 2464 may store instructions 2466. The instructions 2466 may include instructions that, when executed by the processor 2460, cause the processor 2460 to perform the operations described herein. Instructions 2466 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 2468 can include any electronic circuitry and/or logic circuitry to facilitate direct or indirect communication of data between the processor circuit 2450, and other processors or devices. In that regard, the communication module 2468 can be an input/output (I/O) device. In some instances, the communication module 2468 facilitates direct or indirect communication between various elements of the processor circuit 2450 and/or the system 500. The communication module 2468 may communicate within the processor circuit 2450 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the leaflet puncture and slitting device) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles) , 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

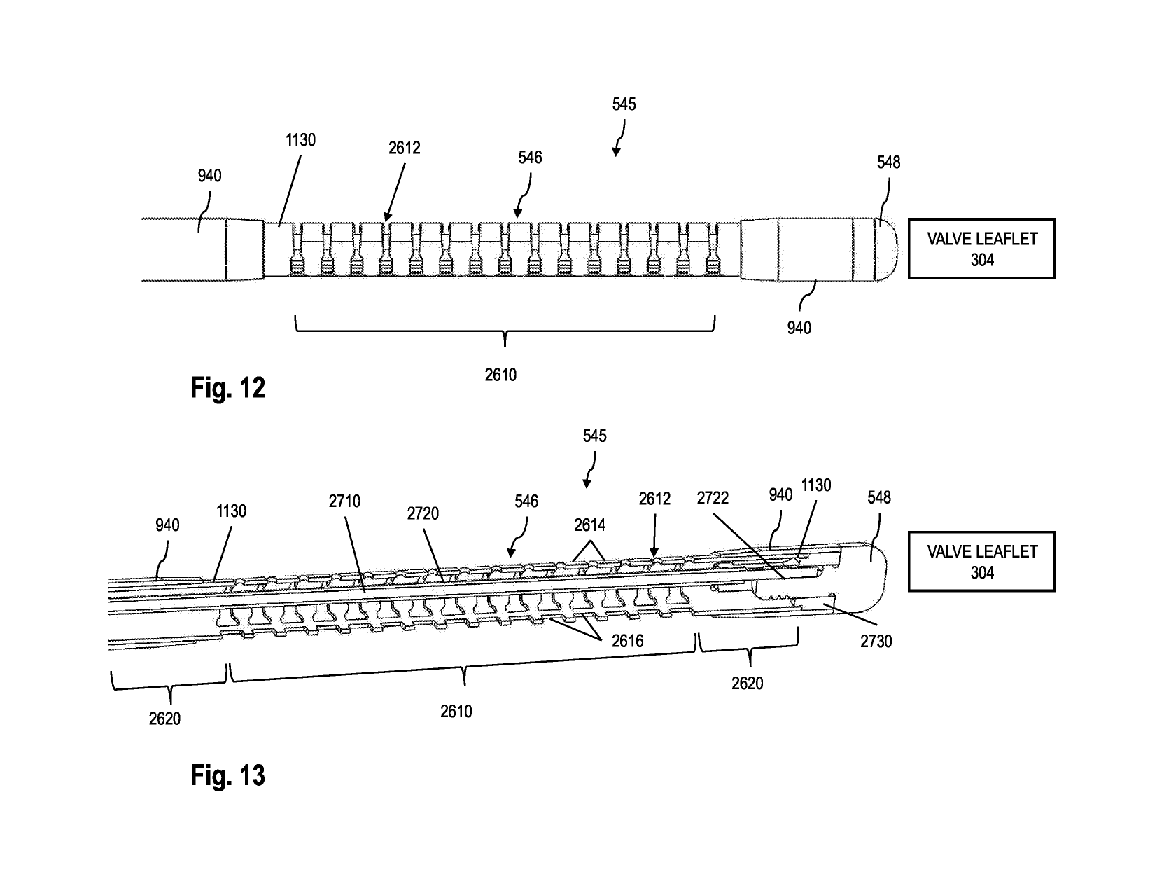

FIG. 12 is a side view of a distal portion of an example resection wire or device 545 in a straight configuration, according to aspects of the present disclosure. The resection wire or device 545 can also be referred to as a flexible elongate intracardiac device that extends through blood vessel to reach the heart or surrounding anatomy (e.g., heart valves, heart chambers, aorta, etc.). Visible are the cutting electrode 546 and the crossing electrode 548. The crossing electrode 548 can also be referred to as a piercing electrode. The intended anatomical targets for the cutting electrode and the crossing electrode 548 include structural heart anatomy, such as aortic valve leaflets 304.

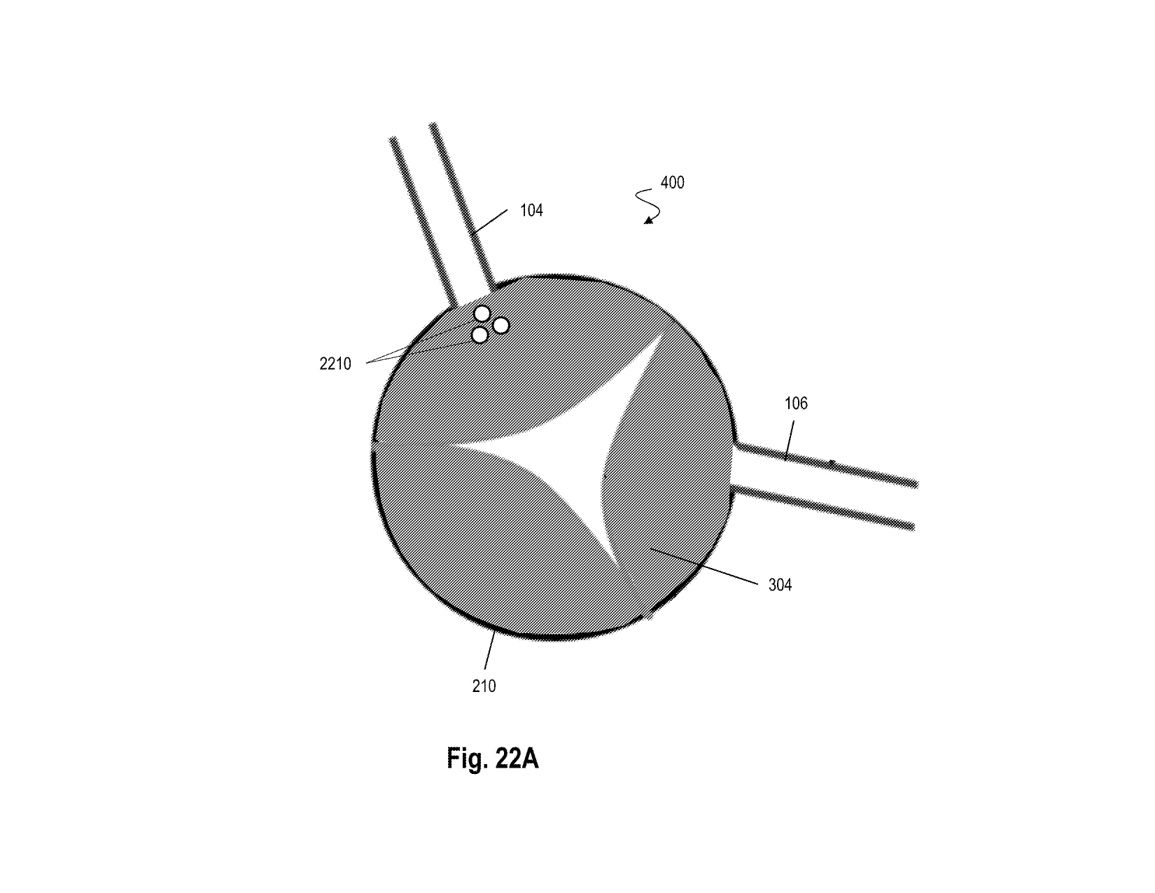

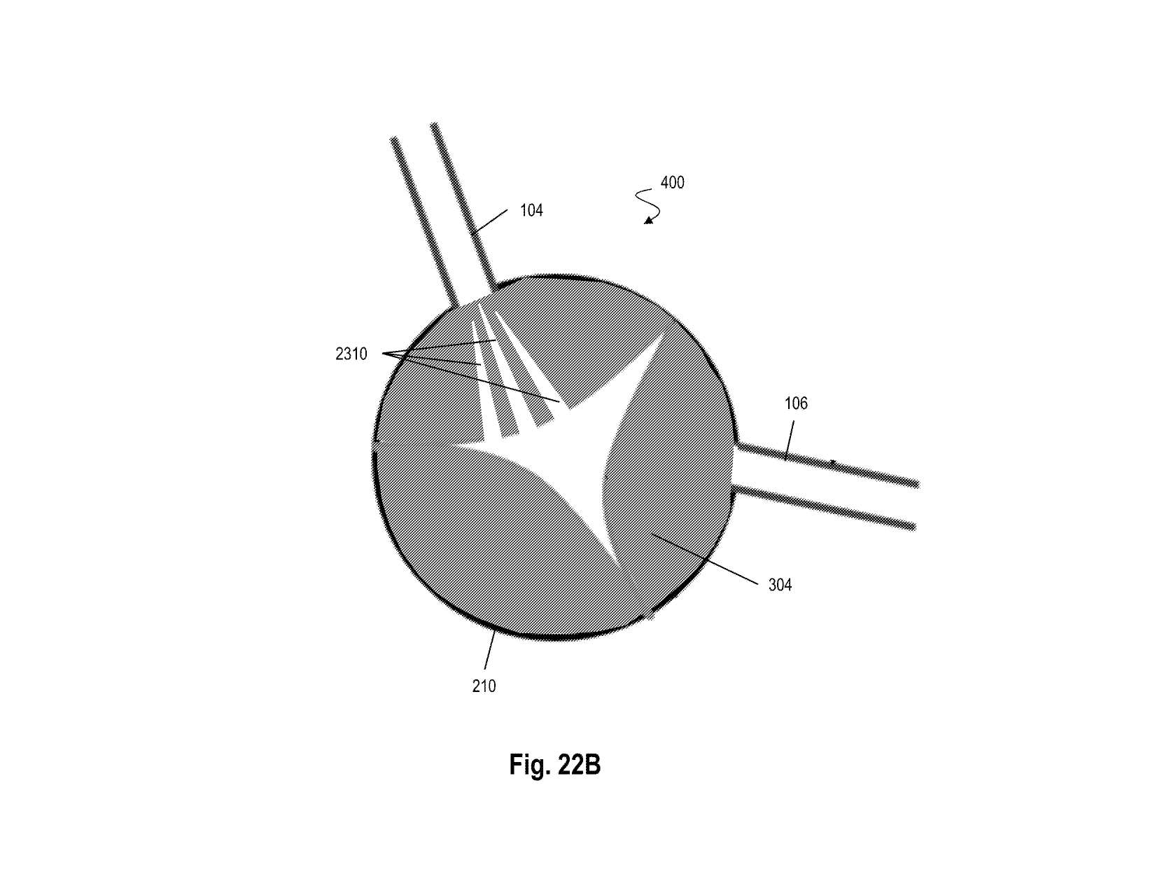

The crossing electrode 548 is positioned at, coupled to, forms, and/or otherwise defines the distal tip of the resection device 545. The crossing electrode 548 is made of a conductive metal or metal alloy. The distal portion of the crossing electrode 548 is uncovered and exposed to the patient’s anatomy. In the straight configuration shown in FIG. 26, the crossing electrode 548 can be energized and contacted with the leaflet 304 to pierce the leaflet 304 (e.g., at a base or other locations of the leaflet), as similarly shown and described with respect to, e.g., FIGS. 22A, 22B, and/or 23A. Accordingly, the straight configuration of FIG. 26 can also be referred to a piercing configuration or a crossing configuration.

The resection device 545 includes the hypotube 1130. The hypotube 1130 can also be referred to as a conductive hypotube 1130 because, as described herein, the hypotube 1130 can carry electrical energy. In some examples, the hypotube 1130 extends for a majority of the length of the resection device 545, such as between the proximal portion and the distal portion of the resection device 545. The hypotube 1130 can include a cut portion 2610. In some aspects, the entire length of the hypotube 1130 is the cut portion 2610. In other example, the hypotube 1130 includes both one or multiple cut portions 2610 and one or multiple uncut portions.

The cutting electrode 546 is formed by, includes, and/or is otherwise defined by the cut portion 2610 of the hypotube 1130. The cut portion 2610 includes cuts 2612 between segments of the hypotube 1130. The cuts 2612 can also be referred to as spaces or gaps. The cut portion 2610 can be cut by laser cutting other suitable types of cutting, such as wire electrical discharge machining (wire EDM). While the portion 2610 is referred to as a cut portion 2610, in other aspects, other types of manufacturing (e.g., 3D printing), are used to form the portion 2610 with the spaces 2612. As described in more detail herein (such as FIG. 33), the cuts 2612 can be different sizes (e.g., widths) at the top and bottom of the hypotube 1130. For example, at the top in FIG. 26, the gaps 2612 are relatively smaller, and at the bottom in FIG. 26, the gaps 2612 are relatively larger.

The cutting electrode 546 is made of a conductive metal or metal alloy. The cutting electrode 546 is uncovered and exposed to the patient’s anatomy. However, when piercing or crossing the leaflet 304 in the straight configuration of FIG. 26, electrical energy is not delivered to the hypotube 1130 (and thus the cutting electrode 546), such that the cutting electrode 546 is not energized to cut the leaflet 304. As described in more detail herein, the cutting electrode 546 is energized when the resection device 545 is in the hooked configuration for cutting the leaflet 304.

The resection device 545 includes different sections of outer insulation layer 940. The outer insulation layer 940 can be one or multiple polymers that provide electrical isolation for the crossing electrode 548 and/or the cutting electrode 546. One section of the outer insulation layer 940 is located proximal of the crossing electrode 548 and the distal of the cutting electrode 546 (e.g., between the crossing electrode 548 and the cutting electrode 546). This section of the outer insulation layer 940 can surround a proximal end of the crossing electrode 548 and the electrical/mechanical connections between a pull wire conductor 2710 and the crossing electrode 548 (shown and described with respect to FIG. 27). Another section of the outer insulation layer 940 is located proximal of the cutting electrode 546. This portion of the outer insulation layer 940 surrounds the portion of the hypotube 1130 that is proximal of the cut portion 2610 such that cutting is only performed by the cutting electrode 546 when the hypotube 1130 is energized.

FIG. 13 is a side cross-sectional view of the resection device 545 of FIG. 26, according to aspects of the present disclosure. Visible are the outer insulation layer 940, cutting electrode 546, crossing electrode 548, hypotube 1130, laser-cut section 2610, and uncut sections 2620.

Also visible is a conductive pullwire or pullwire conductor 2710 surrounded by an inner layer of insulation 2720. In the example shown in FIG. 27, the distal end of the conductive pullwire 2710 (a portion not surrounded by the inner layer of insulation 2720) is electrically coupled and mechanically coupled (e.g., welded, soldered, adhered with a conductive adhesive, etc.) directly to the proximal end of crossing electrode 548 at a location or length 2722. The crossing electrode 548 is electrically isolated from the hypotube 1130 by an insulator 2730, but is electrically connected to the pullwire 2710, which acts as an electrical conductor to energize the crossing electrode 548, for piercing of the valve leaflet 304.

The cross-sectional view of the cut portion 2610 of the hypotube 1130 includes multiple segments 2614 that are separated by gaps 2612. As shown and described with respect to FIGS. 28A and 28B, the gaps 2612 allow the segments 2614 of the cut portion 2610 to be angled relative to another (in response to tension applied to the pullwire conductor 2710 when pulled) so that the cutting electrode 546 transitions from the straight configuration (e.g., FIGS. 26 and 27) to the hook-shaped configuration (e.g., FIGS. 28A and 28B). The cut portion 2610 also has connectors 2616, which are uncut segments of the hypotube 1130. The connectors 2616 physically connect the segments 2614 to one another. The physical continuity provided by the connectors 2616 to the cut portion 2610 allow for all of the cutting electrode 546 to be energized, such as when the hypotube 1130 as a whole is energized. In the example shown in FIG. 27, all of the connectors 2616 are located on one side of the resection device 545 (bottom in drawing). As shown and described with respect to FIGS. 28A and 28B, the connectors 2616 are positioned on the outer portion when the cutting electrode 546 is in the hook-shaped configuration.

In other examples, the resection device 545 can include a catheter shaft with a dedicated lumen for the pullwire 2710. The crossing electrode 548 can be bonded to such a catheter shaft.

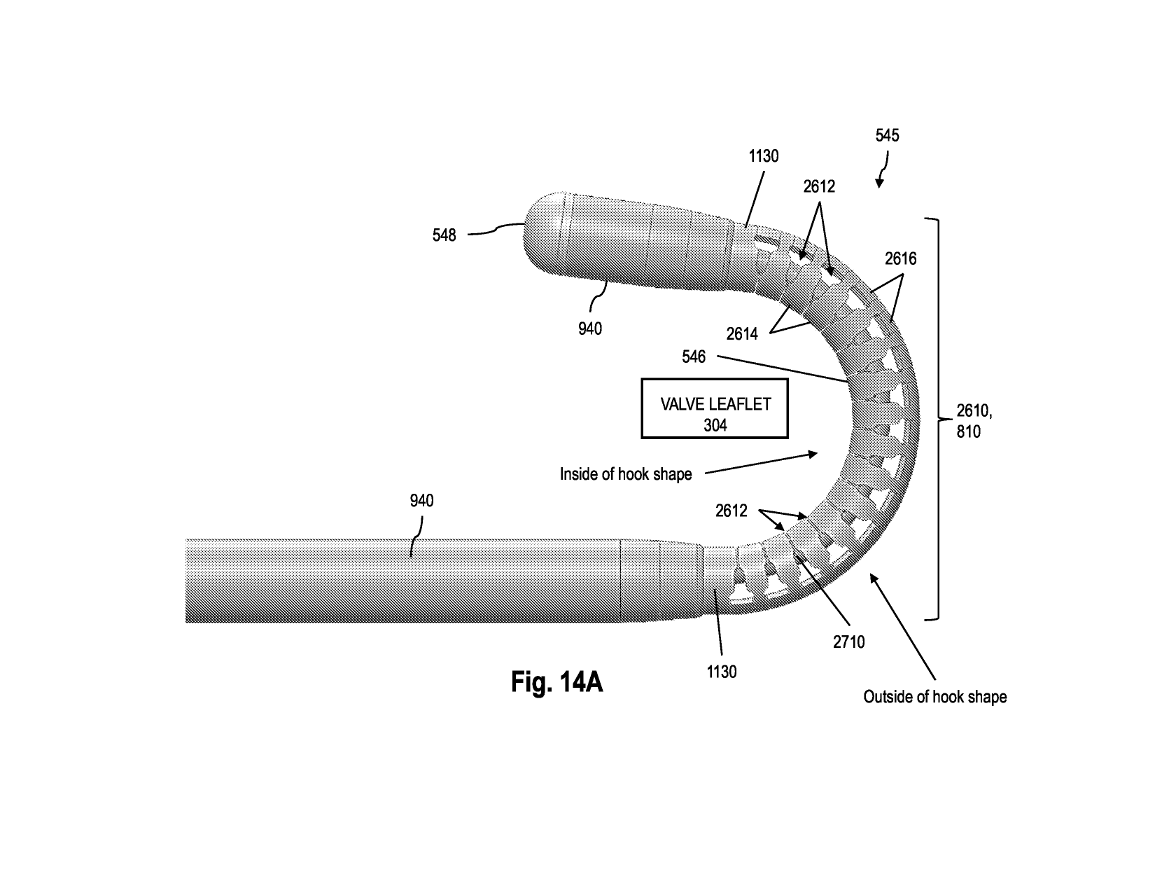

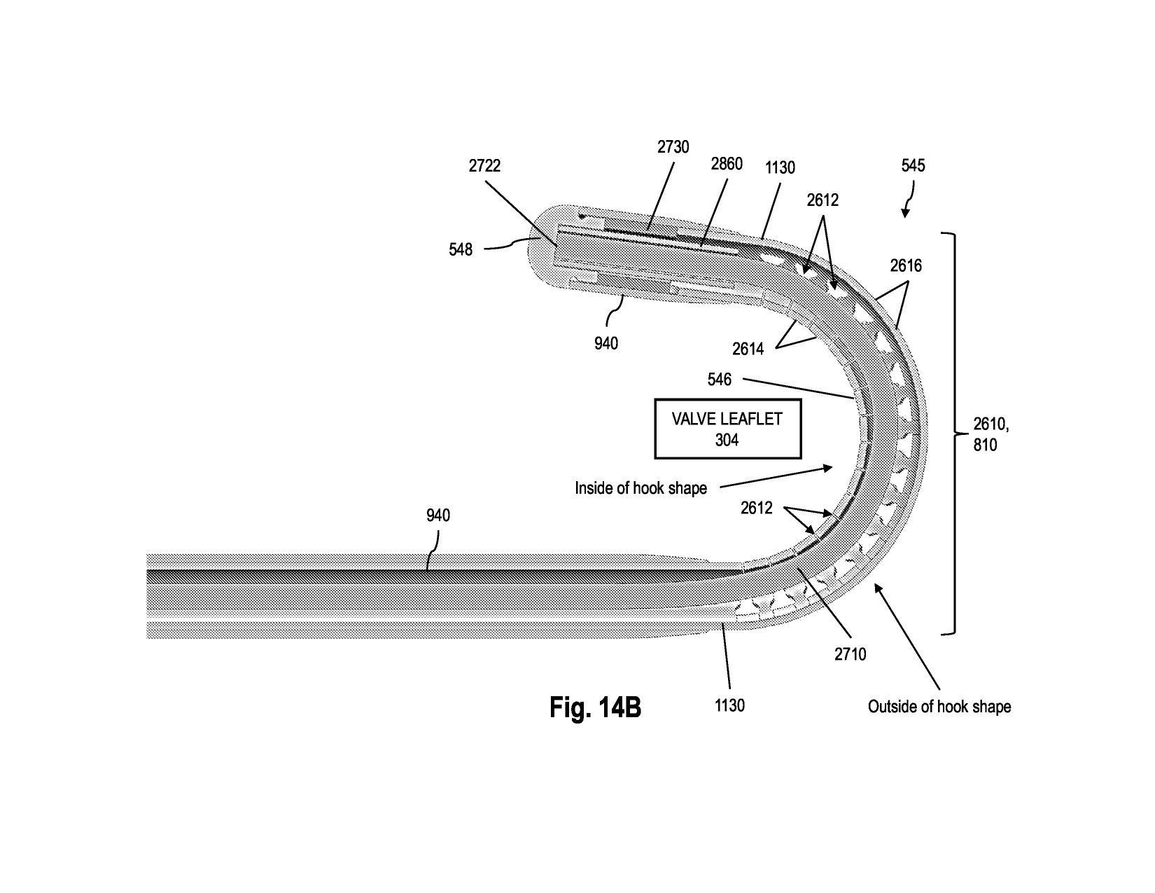

FIG. 14A is a side view of the resection device 545 of FIG. 26 in the hooked configuration, according to aspects of the present disclosure. Visible are the outer insulation layer 940, cutting electrode 546, crossing electrode 548, hypotube 1130, cut section 2610, and pullwire 2710 (visible through gaps 2612). After the crossing electrode 548 has pierced the valve leaflet 304, proximal tension on the pullwire (when the pullwire is pulled in the proximal direction) 2710 has pulled the cut section 2610 into a hook shape 810. The cutting electrode 546 can be pulled through the valve leaflet 304 in order to slit or resect it, as shown and described above. In particular, the portion of the valve leaflet 304 that is proximate to the inside of the hook shape 810 is slit when the resection device 545 is pulled in the proximal direction (causing the energized cutting electrode 546 to move in the proximal direction through this portion of the valve leaflet 304).

In the example shown in FIG. 28A, the portion of the hypotube 1130 that includes the cutting electrode 546 is laser cut or otherwise cut, forming a cut section 2610 that is flexible in such a way that when actuated by the pullwire 2710, cut section 2610 reversibly folds into a hook shape 810 that is rigid as long as tension is maintained on the pullwire 2710. The cutting electrode 546 forms the top of the hook shape via the cut section 2610 and is proximal-facing in the hook-shaped configuration (compared to distal facing in the straight configuration of FIGS. 26 and 27). With the cutting electrode 546 in the hook-shaped configuration, the cutting electrode 546 can be energized and the resection device 545 can be pulled proximally in order to resect the leaflet 304 as described above. Once the resection is complete, tension on the pullwire conductor 2710 is released. The cutting electrode 546 and/or the resection device 545 transitions from the hook-shaped configuration to the straight configuration, and the device 545 is extracted from the patient body, e.g., via the delivery sheath or guide catheter 544 (see FIG. 7A).

The laser cut hypotube 1130 is advantageously flexible yet maintains other desirable properties of a hypotube (e.g., a rigid profile and good torque transmission). In some existing devices, structures at the distal end of a catheter are often created using nitinol super-elastic metal. The disadvantage of such structures is a need to carefully balance forces, between structures being too rigid to fit in a delivery sheath on the one hand, and on the other hand not being rigid enough to perform the desired function. An advantage of the laser-cut hypotube in that when it is activated by a pullwire, it curls into a hook shape of a desired radius. As long as sufficient tension is maintained on the pullwire, the resulting hook shape is rigid enough for tissue cutting application and other applications where a rigid hook shape may be desirable. The process is reversible, e.g. by releasing tension on the pullwire, at which time the cut hypotube can be easily straightened again for extraction.

Deflection into the hooked-shaped configuration of the cutting electrode 546 and/or the resection device 545 can cause the segments 2614 of the cut portion 2610 to be closer to one another on the inside of the hook shape 810 and farther away from one another on the outside of the hook shape 810. In some aspects, the edges of the segments 2614 on the inside of the hook shape 810 can be in contact with another in the hook-shaped configuration. In other aspects, the edges of the segments 2614 on the inside of the hook shape 810 are still spaced from one another in the hook-shaped configuration, but the spacing is less than in the straight configuration (FIGS. 26 and 27). The spacing between the segments 2614 in the hooked shaped configuration can depend on the amount of tension on the pullwire conductor 2710 (more tension results in less spacing, less tension results in more spacing). The connectors 2616 between segments 2614 are the outside of the hook shape 810.

FIG. 14B is a side cross-sectional view of the resection device 545 of FIG. 26 in the hooked configuration, according to aspects of the present disclosure. Visible are the outer insulation layer 940, cutting electrode 546, crossing electrode 548, hypotube 1130, cut section 2610, and pullwire conductor 2710. The insulator 2730 electrically isolates the cutting electrode 546 and the crossing electrode 548 from one another. The pullwire conductor 2710 is attached to the crossing electrode 548 such that it can be used as a conductor to energize the crossing electrode 548, and such that when tension is applied to the pullwire conductor 2710, the cut section 2610 of the hypotube 1130 forms the hook shape 810, with the cutting electrode 546 on the inside of the hook shape.

In the example shown in FIG. 28B, once the crossing electrode 548 has pierced the valve leaflet 304, tension on the pullwire conductor 2710 has pulled the laser-cut section 2610 into a hook shape 810, such that the cutting electrode 546 can be pulled through the valve leaflet 304 in order to slit or resect it, as shown and described above.

In the example of FIGS. 28A and 28B, the resection device 545 includes one pullwire conductor 2710. The one pullwire conductor 2710 can allow for one type of deflection for the distal portion of the resection device 545. In particular, the cutting electrode 546 and/or the resection device 545 deflects in one direction when tension is applied to the pullwire conductor 2710. As shown in FIG. 27, 28A, and 28B, for example, a longitudinal axis of the pullwire conductor 2710 and/or the coupling location 2722 between the pullwire conductor 2710 and the crossing electrode 584 can be positioned so that it is radially offset from the central longitudinal axis of the resection device 545 and/or the hypotube 1130 (e.g., radially positioned towards the inside of the hook shape). The pullwire conductor 2710 and/or coupling location 2722 being radially offset allows for the deflect when tension is applied to the pullwire conductor 2710. As such, the cut section 2610 of the hypotube 1130 flexes readily to form the hook shape 810, but does not easily flex in the in the direction opposite the hook, or out of the plane of the hook, such that when tension is applied to the pullwire 270, the cut section 2610 flexes to form the hook shape 810 with the cutting electrode 546 on the inside of the hook shape, rather than an outside of the hook shape. The one direction of deflection results in, for example, the side of the cut portion 2610 with less spacing in the straight configuration (top side in FIGS. 26 and 27) becomes the inside of the hook shape 810, the side of the cut portion 2610 with more spacing in the straight configuration (bottom side in FIGS. 26 and 27) becomes the outside of the hook shape 810 side, and the connectors 2616 between segments 2614 are on the outside of the hook shape 810. The cross-sectional view plane in FIG. 28 extends through the connectors 2616, which shows the physical continuity that that connectors 2616 provide to the cut portion 2610.

The arrangement of components at the distal portion of resection device 545 has some differences between FIG. 28B and FIG. 27. In FIG. 28B, the proximal end of crossing electrode 548 has an opening that receives the distal end of the pullwire conductor 2710. The pullwire conductor 2710 can be mechanically and electrically coupled to the crossing electrode 548 inside of the opening at the location 2722. In FIG. 27, the proximal end of crossing electrode 548 is shaped as an extension. Thus, the distal end of the pullwire conductor 2710 extends alongside the proximal end of crossing electrode 548 at the location 2722 where the pullwire conductor 2710 and the crossing electrode 548 are coupled in FIG. 27. An end hypotube 2860 is crimped, friction-fit, or soldered to the pullwire conductor 2710 and welded or soldered to the crossing electrode 548, to form both the electrical and mechanical connections between the pullwire conductor 2710 and the crossing electrode 548.

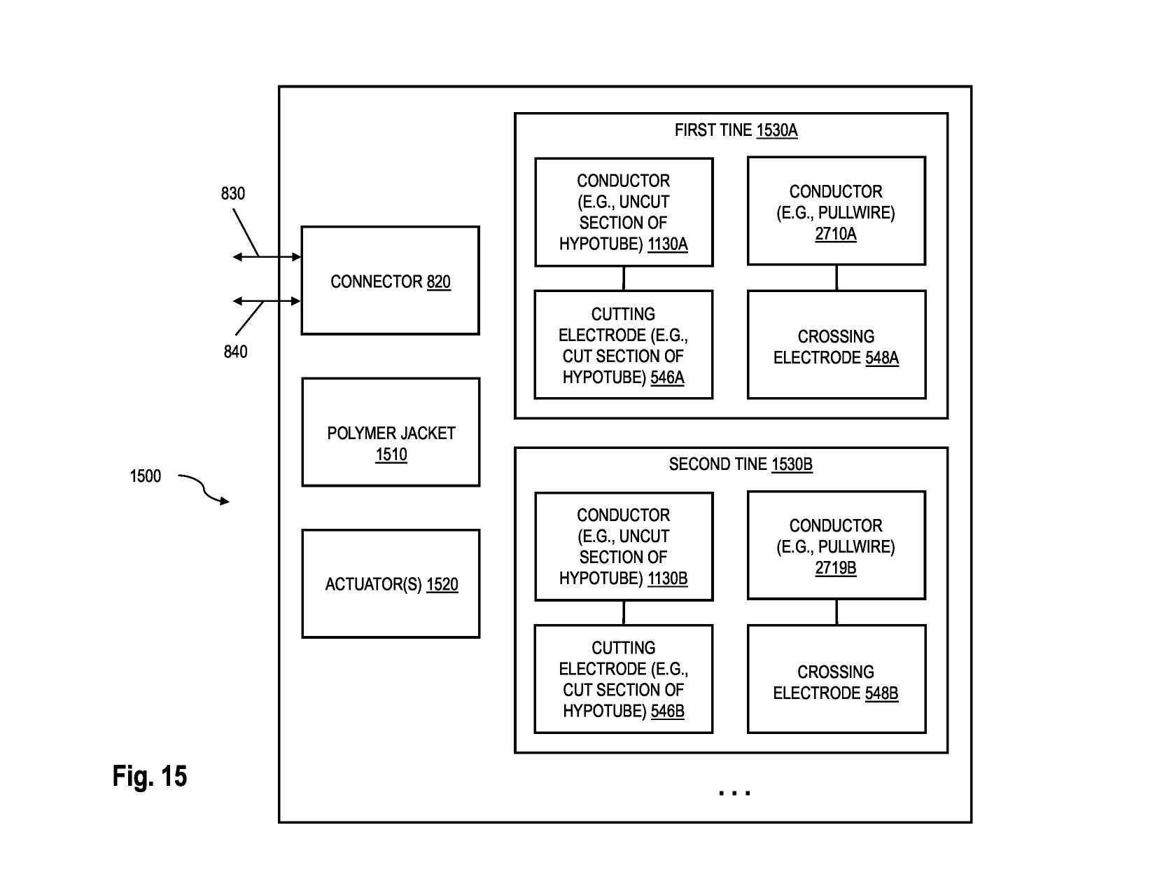

FIG. 15 is a schematic, diagrammatic representation of a multi-tine tissue resection wire 1500, according to aspects of the present disclosure. The multi-tine resection wire includes a first tine 1530A and a second tine 1530B, partially enclosed in a polymer jacket 1510 and with bending controlled by actuators 1520. The cutting electrode conductor 1130A of the first tine 1530A is in electrical communication with conductors 830, 840 of the connector 820, and with the cutting electrode 546A of the first tine 1530A. The crossing electrode conductor or pullwire 2710A is in electrical communication with the conductors 830, 840 of the connector 820, and with the crossing electrode 548A of the first tine 1530A. Similarly, the cutting electrode conductor 1130B of the second tine 1530B is in electrical communication with conductors 830, 840 of the connector 820, and with the cutting electrode 546B of the second tine 1530B, and the crossing electrode conductor or pullwire 2710B is in electrical communication with the conductors 830, 840 of the connector 820, and with the crossing electrode 548B of the second tine 1530B.

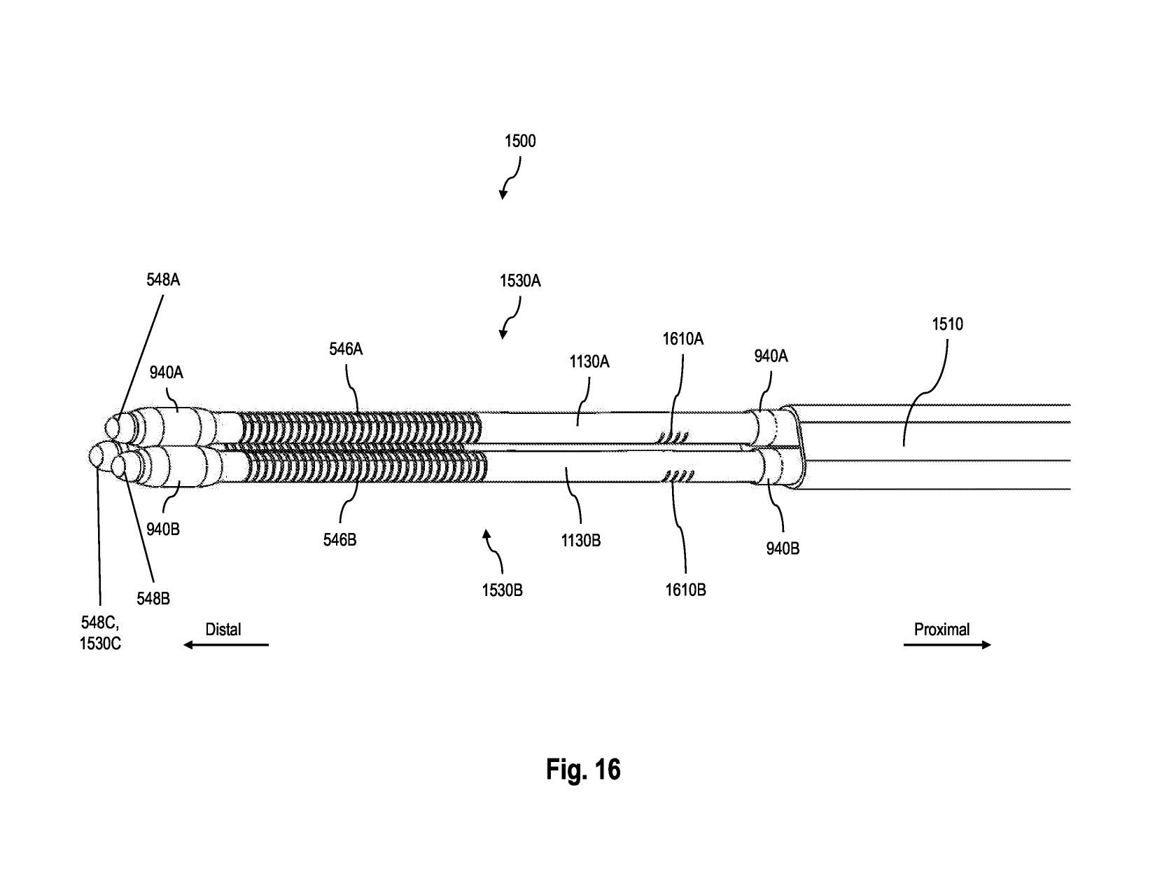

In some aspects, there may be only two tines, 1530A and 1530B. In other aspects, there may be three, four, or more tines. Exemplary aspects with three tines are shown below in FIGS. 16 and 18-21.

FIG. 16 is a schematic, diagrammatic, perspective view of a three-tine tissue resection wire 1500 in its straight configuration, according to aspects of the present disclosure. Visible are the polymer jacket 1510, first tine 1530A, second tine 1530B, and third tine 1530C. Also visible are the piercing electrodes or crossing electrodes 548A, 548B, and 548C of the first, second, and third tines, respectively. Also visible are the insulator 940A, cutting electrode 546A, and cutting electrode conductor 1130A of the first tine. Also visible are the insulator 940B, cutting electrode 546B, and cutting electrode conductor 1130B of the second tine. Also visible are cut portions 1610A and 1610B, which facilitate bending of the first and second tines 1530A, 1530B. The cut portions 1610A and 1610B are located proximal of the cutting electrodes 546A and 546B, and distal of the polymer jacket 1510, in the portion of the times 1530A and 1530B not covered by the insulation layers 940A and 940B.

The first tine 1530A, second tine 1530B, and the third tine 1530C can be made of a metal or metal alloy, such as nitinol or other metal/metal alloy with super elastic properties. As described herein, the first tine 1530A, second tine 1530B, and the third tine 1530C can transition between a straight configuration and a hooked shaped configured by actuation (pulling/releasing) of an internal pull wire (e.g., pull wire conductor). In other aspects, the first tine 1530A, second tine 1530B, and the third tine 1530C can be pre-shaped or pre-set into the hook-shaped configuration such that, when the tines automatically return to hook-shaped configuration after being put into a straight configuration (e.g., tines put into straight configuration inside of a lumen of, e.g., a guide catheter, then move tines out of lumen, which allows them to return to hook-shaped configuration).

The polymer jacket 1510 keeps the proximal portions of the three tines clustered together. Distal of the jacket 1510, the tines 1530A, 1530B, etc. are free to bend independently of one another, e.g., at the cut portions 1610A, 1610B, and at the cutting electrodes 546A and 546B. The polymer jacket 1510 may for example be made of a flexible insulating material such as a polymer. The polymer jacket 1510 defines a lumen inside of which the multiple tines are positioned. However, the polymer jacket 1510 is not loosely positioned around the multiple tines, which would to allow rotational and/or longitudinal motion of the multiple tines relative to one another and/or the jacket itself. Rather, in an example, the polymer jacket 1510 is reflowed (melted and then cured) with polymer insulation layer 940A, 940B, 940C of the multiple tines 1530A, 1530B, 1530C, which bonds/blends the polymers together. This mechanically fixes these components to one another, preventing longitudinal motion and rotational motion of the multiple tines 1530A, 1530B, 1530C relative to one another and/or the polymer jacket 1510 itself. In an example, the polymer jacket 1510 is tightly/securely positioned around the first tine 1530A, the second tine 1530B, and the third tine 1530C. In a different aspect, the jacket can be looser so that the multiple tines are allowed to have rotational and/or longitudinal motion relative to one another and/or the jacket itself.

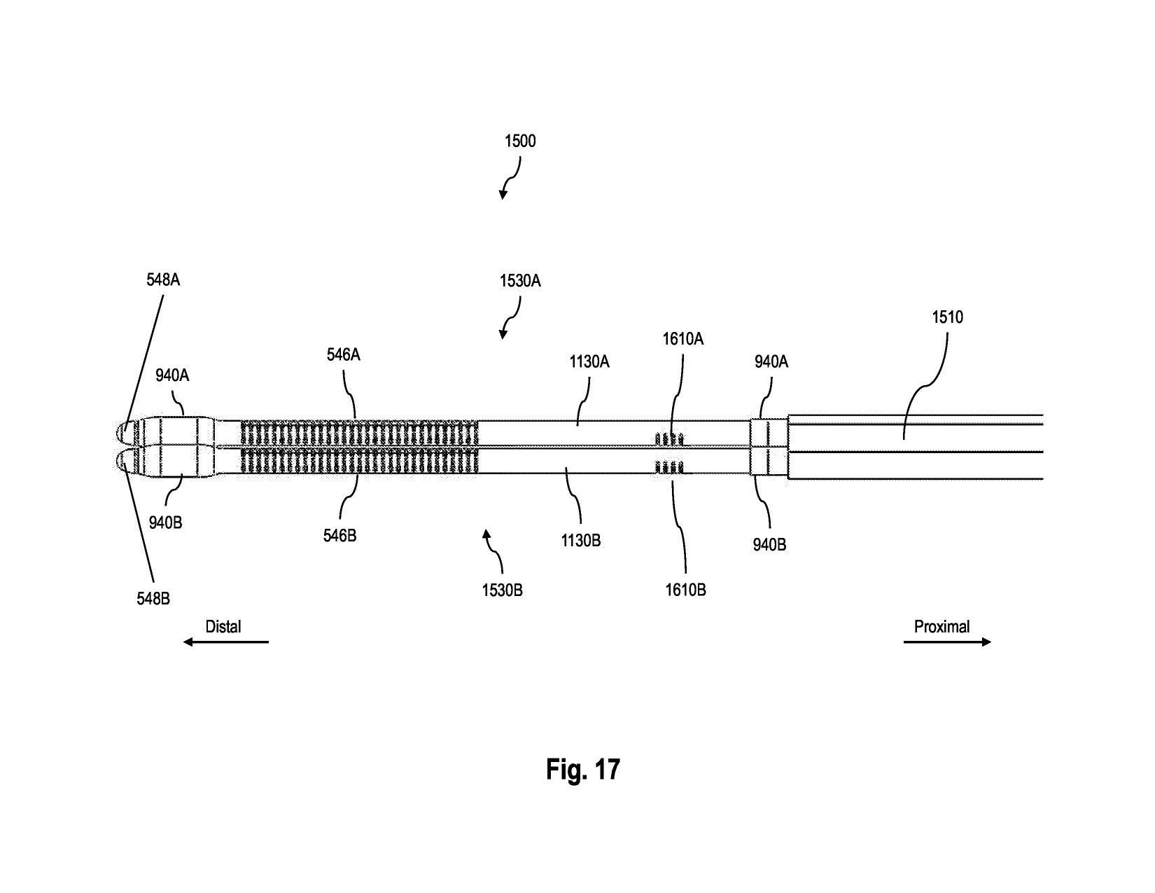

FIG. 17 is a schematic, diagrammatic side view of a multi-tine tissue resection wire 1500, having at least two tines, in their straight configuration, according to aspects of the present disclosure. Visible are the polymer jacket 1510, first tine 1530A, and second tine 1530B. Also visible are the piercing electrodes or crossing electrodes 548A, 548B, and of the first and second tines, respectively. Also visible are the insulator 940A, cutting electrode 546A, and cutting electrode conductor 1130A of the first tine 1530A. Also visible are the insulator 940B, cutting electrode 546B, and cutting electrode conductor 1130B of the second tine 1530B. Also visible are cut portions 1610A and 1610B, which facilitate bending of the first and second tines 1530A, 1530B.

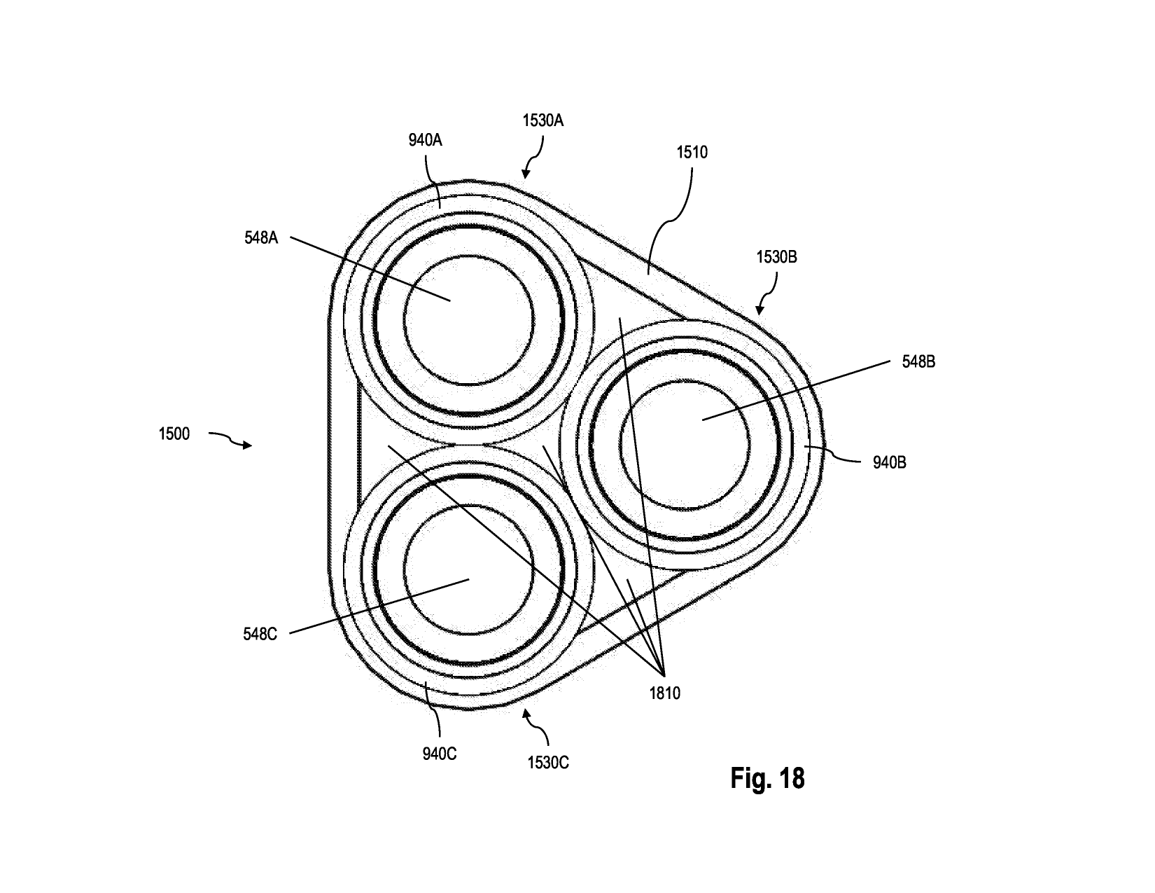

FIG. 18 is a schematic-diagrammatic end view of a three-tine tissue resection wire 1500 in its straight configuration, according to aspects of the present disclosure. Visible are the polymer jacket 1510, first tine 1530A, first tine insulator 940A, and first tine crossing electrode 548A. Also visible are the second tine 1530B, second tine insulator 940B, second tine crossing electrode 548B, third tine 1530C, third tine insulator 940C, and third tine crossing electrode 548C.

In the example shown in FIG. 18, three tines 1530A, 1530B, 1530C are arranged in a triangle within a lumen 1810 of the polymer jacket 1510. This may be done for example to minimize the cross-sectional area of the resection wire 1500 so that it fits more easily through the tortuous vasculature of the patient. In ither aspects, there may for example be two tines arranged side-by-side, four tines arranged in a square, or otherwise.

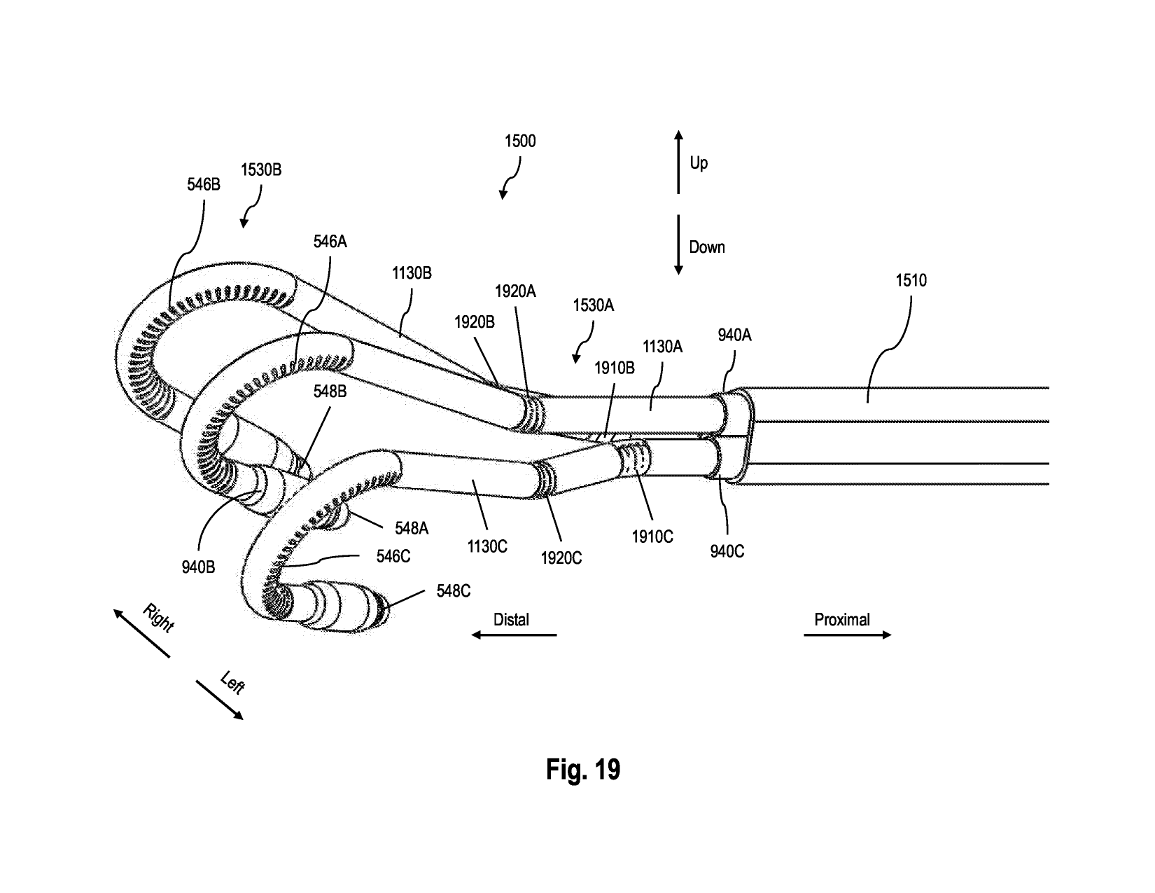

FIG. 19 is a schematic, diagrammatic perspective view of a three-tine tissue resection wire 1500 in its hooked configuration, according to aspects of the present disclosure. Visible are the polymer jacket 1510, first tine or center tine 1530A, second tine or right tine 1530B, and third tine or left tine 1530C. Also visible are the cutting electrode conductors 1130A, 1130B, 1130C, cutting electrodes 546A, 546B, 546C, insulators 940A, 940B, 940C, and crossing electrodes 548A, 548B, and 548C.

Also visible are cut portions 1910B and 1910C, which enable the second tine 1530B and third tine 1530C to bend away from the first tine or center tine in the right and left directions, respectively. In addition, cut portions 1920A, 1920B, and 1920C allow the tines 1540A, 1530B, and 1530C to bend in an upward direction. The cut portions 1920A, 1920B, 1920C are positioned distal of the cut portions 1910B, 1910C, and proximal of the cutting electrodes 546A, 546B, and 546C. In an example, the tines 1530A, 1350B, 1530C are made of a shape-memory alloy such as Nitinol, and this bending occurs automatically (e.g., without actuators) when the tines are extended from the delivery sheath or guide catheter 544 (see FIG. 7A), and reverses automatically when the tines are retracted back into the delivery sheath or guide catheter 544. The deflection of the cutting electrodes 546A, 546B, and 546C from their straight configuration (shown in FIGS. 18-20) into their hooked configuration (shown here) may be under the control of a single actuator 1520 (e.g., controlling all three tines), two actuators 1530 (e.g., one actuator 1520 controlling the center tine and one controlling the left and right tines), or three actuators (each controlling a separate tine). Other arrangements are possible, and fall explicitly within the scope of the present disclosure.

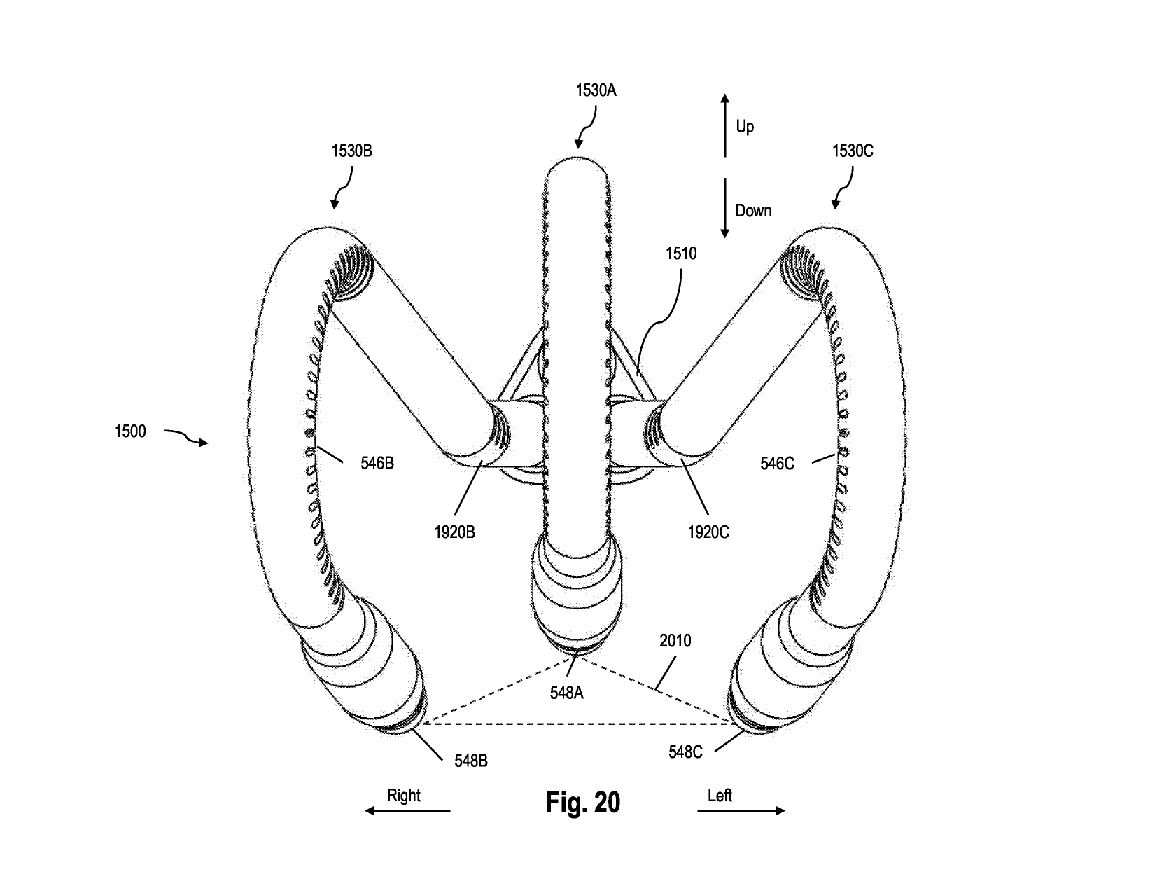

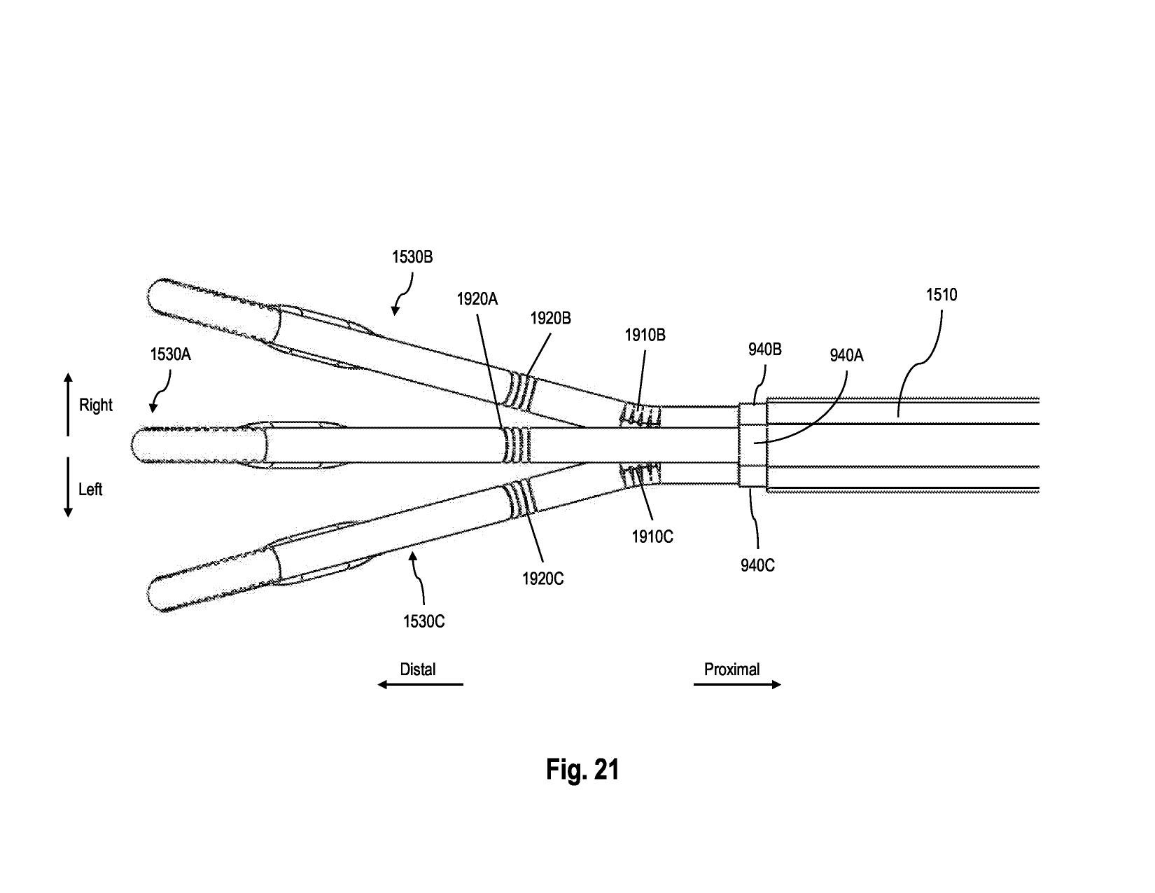

FIG. 20 is a schematic, diagrammatic end view of an example three-tine resection wire 1500 in its hooked configuration, according to aspects of the present disclosure. Visible are the polymer jacket 1510, center tine 1530A, right tine 1530B, left tine 1530C, cut portions 1920B and 1920C, cutting electrodes 546B and 546C, and crossing electrodes or piercing electrodes. In the example shown in FIG. 20, the crossing electrodes 548A, 548B, and 548C form a triangle 2010, with the center crossing electrode 548A sitting higher than the right crossing electrode 548B and left crossing electrode 548C. In other aspects, depending on the implementation, the crossing electrodes 548A, 548B, 548C may form a straight line, or else a triangle with the center crossing electrode 548A positioned lower than the left and right crossing electrodes.

FIG. 21 is a schematic, diagrammatic top view of an example three-tine resection wire 1500 in its hooked configuration, according to aspects of the present disclosure. Visible are the polymer jacket 1510, insulators 940A, 940B, 940C, center tine 1530A, right tine 1530B, left tine 1530C, and cut regions 1910B, 1910C, 1920A, 1920B, and 1920C.

It is noted that the right and left tines, 1530B and 1530C, are the same as one another and are different from the center tine 1530A. Specifically, the cut regions 1910B and 1910C allow the right tine 1530B to bend in a rightward direction away from the center tine 1530A and allow the left tine 1530C to bend in a leftward direction away from the center tine 1530A. The center tine 1530A does not have a cut in the position corresponding to cuts 1910B and 1910C. However, in all three tines, the cut regions 1920A, 1920B, 1920C allow the tines to bend in an upward direction (see FIGS. 19 and 20), whether by the same amount or different amounts.

FIG. 22A is a diagrammatic top view of an aortic valve 400, according to aspects of the present disclosure. Visible are the heart wall 210, the aortic valve leaflets 304, the right coronary artery 104, and the left main coronary artery 106. During and after valve replacement procedures, the leaflets 304 can obstruct the arteries 104, 106, as described above. This and other issues can, in certain circumstances, be alleviated by resecting one or more leaflets 304. Thus, in the example shown in FIG. 22A, one leaflet has been given three puncture lumens 2210 (e.g., by a three-tine leaflet puncture and slitting device whose crossing electrodes have been energized and passed through the leaflet 304). The multiple crossing electrodes simultaneously puncture the heart valve leaflet during an individual movement of the intracardiac device. The individual movement can be a forward longitudinal movement of the leaflet puncture and slitting device while multiple tines are in the straight configuration. For example, the doctor pushes the device, and therefore the multiple tines forward, into contact with the leaflet. Because the crossing electrodes are energized and the tines are in the straight configuration, pushing the device/multiple tines into contact with the leaflet causes the crossing electrodes to simultaneously puncture the leaflet.

FIG. 22B is a diagrammatic top view of an aortic valve 400, according to aspects of the present disclosure. Visible are the heart wall 210, the aortic valve leaflets 304, the right coronary artery 104, and the left main coronary artery 106. In the example shown in FIG. 22B, one leaflet has been given three total resection cuts 2310 (e.g., by a three-tine leaflet puncture and slitting device 700 whose hook-shaped cutting electrodes are pulled along the length of the leaflet 304 from the puncture lumens 2210 to the leaflet tip). The multiple cutting electrodes simultaneously slit the heart valve leaflet during an individual movement of the intracardiac device. The individual movement can be a backward longitudinal movement of the leaflet puncture and slitting device while multiple tines are in the hook-shaped configuration. For example, the doctor pulls the device back, and therefore the multiple tines back. Because the cutting electrodes are energized, the tines are in the hook-shaped configuration, and at least a part of the leaflet is captured/hooked inside of the hook-shape configuration, pulling the device back causes the cutting electrodes to simultaneously slit the leaflet.

As will be readily appreciated by those having ordinary skill in the art after becoming familiar with the teachings herein, the multi-tined leaflet puncture and slitting device advantageously permits a clinician (e.g., a heart surgeon) to produce multiple punctures and slits in a heart valve leaflet with a repeatably straight incisions that cut away from the heart wall and thus present minimal risk of damage to the heart wall or other adjacent tissues. Although the electrical energy powering the multiple crossing and cutting electrodes is described herein as being radio frequency (RF) energy, it is understood that other types of electrical energy may be used instead or in addition, including but not limited to terahertz, gigahertz, microwave, 120 Hz, 60 Hz, 0 Hz (e.g., direct current or DC), etc.

Although intended for specific treatment of heart valve related diseases, the technology disclosed herein could be expanded to anywhere tissue needs to be remotely, multiply punctured and then slit, such as endoscopic, laparoscopic, intravascular, intraluminal, and/or robotic surgical procedures. The multi-tined leaflet puncture and slitting device can replace the more complex BASILICA and LAMPOON procedures with a standalone device that requires less training and experience to operate, and that is capable of forming several slits simultaneously. This device is intended for any medical procedure where a thin section of tissue needs to be punctured and then slit multiple times, either partially or completely, where there is access to the area of the tissue via a catheter conduit.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader’s understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word "comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
a flexible elongate member configured to be positioned within a patient body and comprising:
a first tine comprising a first crossing electrode and a first cutting electrode;
a second tine comprising a second crossing electrode and a second cutting electrode; and
a polymer jacket positioned around the first tine and the second tine, wherein the polymer jacket is configured to prevent longitudinal motion and rotational motion of the first tine and the second tine relative to one another,
wherein the first tine and the second tine are configured to transition between a straight configuration and a hook-shaped configuration,
wherein the first crossing electrode and the second crossing electrode are configured to cut anatomy of the patient body while the first tine and the second tine are in the straight configuration,
wherein the first cutting electrode and the second cutting electrode are configured to cut the anatomy while the first tine and the second tine are tine are in the hook-shaped configuration.

2. The apparatus of claim 1,
wherein the first tine and the second tine each comprise a proximal portion and a distal portion,
wherein the polymer jacket is positioned around the proximal portion of the first tine and the second tine.

3. The apparatus of claim 2,
wherein the first crossing electrode and the first cutting electrode are positioned at the distal portion of the first tine,
wherein the second crossing electrode and the second cutting electrode are positioned at the distal portion of the second tine,
wherein the polymer jacket is not positioned around the distal portion of the first tine and the second tine such that the distal portion of the first tine and the second tine are uncovered to allow transition between the straight configuration and the hook-shaped configuration.

4. The apparatus of claim 1, wherein the flexible elongate member comprises a third tine comprising a third crossing electrode and a third cutting electrode.

5. The apparatus of claim 4, wherein the first tine, the second tine, and the third tine are arranged in a triangular configuration.

6. The apparatus of claim 5, wherein the jacket is configured to fixedly maintain the first tine, the second tine, and the third tine in the triangular configuration.

7. The apparatus of claim 4, wherein the polymer jacket is bonded with a polymer of the first tine, a polymer of the second tine, and a polymer of the third tine.

8. The apparatus of claim 1, wherein the first tine and the second tine are more radially spaced from one another in the hooked configuration than in the straight configuration.

9. The apparatus of claim 8, wherein the second tine is configured to have radial motion in a first direction relative to the first tine when the first tine and the second tine transition from the straight configuration to the hooked configuration.

10. The apparatus of claim 9,
wherein the flexible elongate member comprises a third tine configured to transition between the straight configuration and the hook-shaped configuration,
wherein the third tine is configured to have radial motion in an opposite, second direction relative to the first tine when the first tine, the second tine, and the third tine transition from the straight configuration to the hook-shape configuration.

11. The apparatus of claim 10, wherein the first tine comprises a central tine, wherein the second tine is positioned on a first side of the central tine, and the third tine is positioned on an opposite second side of the central tine.

12. The apparatus of claim 1, wherein the first tine and the second tine comprise a different structure from one another.

13. The apparatus of claim 12,
wherein the second tine and not the first tine comprises a first plurality of slits configured to provide radial motion relative to the first tine,
wherein the plurality of slits are positioned proximal of the second crossing electrode and the second cutting electrode.

14. The apparatus of claim 13, wherein the first tine and the second tine comprise a second plurality of slits configured to provide deflection, in a same direction, for:
the first crossing electrode and the first cutting electrode; and
the second crossing electrode and the second cutting electrode.

15. The apparatus of claim 1,
wherein the anatomy comprises a heart valve leaflet,
wherein, to cut the heart valve leaflet, the first crossing electrode and the second crossing electrode are configured to simultaneously puncture the heart valve leaflet,
wherein, to cut the heart valve leaflet, the first cutting electrode and the second cutting electrode are configured to simultaneously slit the heart valve leaflet.

16. The apparatus of claim 15,
wherein the first crossing electrode and the second crossing electrode are configured to simultaneously puncture the heart valve leaflet during an individual first movement of the intracardiac device, and
wherein the first cutting electrode and the second cutting electrode are configured to simultaneously slit the heart valve leaflet during an individual second movement of the intracardiac device.

17. The apparatus of claim 16,
wherein the individual first movement of the intracardiac device comprises a forward longitudinal movement of the intracardiac device while in a straight configuration,
wherein the individual second movement of the intracardiac device comprises a backward longitudinal movement of the intracardiac device while in a hook-shaped configuration.
